(19) United States
(12) Patent Application Publication
(10) Pub. No.: US 2026/0102528 A1
(43) Pub. Date: Apr. 16, 2026

(54) BINDING AGENT RECOGNITION OF DRUG-PEPTIDE CONJUGATES

(71) Applicant: The Regents of the University of California, Oakland, CA (US)

(72) Inventors: Charles S. CRAIK, San Francisco, CA (US); Kevan M. SHOKAT, San Francisco, CA (US); Peter J. ROHWEDER, San Francisco, CA (US); Ziyang ZHANG, San Francisco, CA (US); Chayanid ONGPIPATTANAKUL, San Francisco, CA (US); Michael J. EVANS, Pacifica, CA (US)

(73) Assignee: The Regents of the University of California, Oakland, CA (US)

(21) Appl. No.: 19/109,104

(22) PCT Filed: Sep. 8, 2023
(86) PCT No.: PCT/US2023/073785

§ 371 Date: Mar. 5, 2025

Related U.S. Application Data
(60) Provisional application No. 63/404,869, filed on Sep. 8, 2022.

(51) Int. Cl. A61K 51/10; A61P 35/00; C07K 16/44; G01N 33/94

(52) U.S. Cl. CPC A61K 51/1045; A61P 35/00; C07K 16/44; G01N 33/94; A61K 2121/00; C07K 2317/24; C07K 2317/565

(57) ABSTRACT

The present disclosure relates generally to antibodies reactive with drug-peptide conjugates, as well as drug-peptide-binding fragments thereof. The present disclosure also relates to nucleic acids, expression cassettes, and expression vectors encoding the antibodies and drug-peptide-binding fragments. The antibodies and binding fragments are useful for diagnosis and treatment of cancers that present a neoantigen formed by binding of an inhibitor to an oncoprotein.

### CROSS-REFERENCE TO RELATED APPLICATIONS

This application claims priority to and the benefit of U.S. Provisional Application No. 63/404,869, filed Sep. 8, 2022, the disclosure of which is herein incorporated by reference in its entirety.

### STATEMENT REGARDING FEDERALLY SPONSORED RESEARCH OR DEVELOPMENT

This invention was made with government support under Grant Nos. R01 CA190408 and P41 CA196726 awarded by the National Institutes of Health. The government has certain rights in the invention.

### REFERENCE TO ELECTRONIC SEQUENCE LISTING

The content of the electronic sequence listing (643662002940SEQLIST.xml; Size: 98,595 bytes; and Date of Creation: Sep. 6, 2023) is herein incorporated by reference in its entirety.

### TECHNICAL FIELD

The present disclosure relates generally to antibodies reactive with drug-peptide conjugates, as well as drug-peptide-binding fragments thereof. The present disclosure also relates to nucleic acids, expression cassettes, and expression vectors encoding the antibodies and drug-peptide-binding fragments. The antibodies and binding fragments are useful for diagnosis and treatment of cancers that present a neoantigen formed by binding of an inhibitor to an oncoprotein.

### BACKGROUND

The search for tumor specific antigens has been a longstanding goal in oncology as a means to differentially recognize tumor cells from non-tumor cells. However, there are very few unique cell-surface proteins that cancer cells express that are not expressed on any subset of non-tumor cells, thus limiting the therapeutic index achievable by this method. Most oncogenes are either not overexpressed or are intracellular and thus inaccessible to antibodies. Despite this, the selective modulation of a patient's immune system through cancer immunotherapy has emerged as a powerful therapeutic strategy. This therapeutic shift towards modulation of an entire system of cells has shown great promise, even demonstrating the potential for cures in a subset of patients. The protein and cell-based efforts modulate immune responses through the specific recognition of tumor antigens or immune cell receptors/ligands at the cell surface. These approaches, however, are limited by the aforementioned requirement for extracellular recognition, significantly restricting potential target antigens to those accessible at the cell surface. Additionally, many current immunotherapies target upregulated proteins rather than tumor-specific antigens, resulting in “on-target/off-tumor” toxicities.

One class of molecule almost universally present on the cell surface is the class I major histocompatibility complex (MHC-I). The immune system natively monitors intracellular proteins through antigen presentation on MHC-I. Short peptides (8-11 amino acids) derived from proteasomal degradation of proteins are loaded onto complexes and presented on the cell surface as peptide-MHCs (pMHCs). These complexes serve as a readout of intracellular health and are natively surveyed by T-cells for foreign antigens. Recently, however, the specific targeting of tumor-associated pMHCs with immunotherapy has been found to be a powerful therapeutic strategy (Tran et al., New England Journal of Medicine 375, 2255-2262, 2016; and Dao et al., Nature Biotechnology 33, 1079-1086, 2015). The specific recognition of a pMHC can be achieved with either a T-cell receptor (native or engineered) or an engineered antibody fragment. However, MHC genes are highly polymorphic, with more than 19,000 alleles having been deposited with the International Immunogenetics Information System. T-cells are required to make direct contact with the presenting MHC molecule during immune development, meaning T cell receptors (TCRs) are unlikely to be cross reactive with the same target peptide presented by different MHC molecules. Similarly, while recognition of pMHCs with engineered antibody fragments theoretically does not require the antibody epitope to contain residues of the presenting MHC allele, in practice the antibody binding site is typically not restricted to the peptide component of the pMHC (Chang et al., Expert Opin Biol Ther 16, 979-987, 2016). Therefore, targeting native pMHCs with either TCRs or antibody fragments are strategies that are likely to benefit a small subset of patients in need of immunotherapy.

Accordingly, the art needs tumor-specific therapeutic agents capable of directing an immune response to intracellular oncoproteins in cancer patients. The direct targeting of intracellular oncoproteins would significantly widen the therapeutic window by reducing potential “on-target/off-tumor” side effects.

### BRIEF SUMMARY

The present disclosure relates generally to antibodies reactive with drug-peptide conjugates, as well as drug-peptide-binding fragments thereof. The present disclosure also relates to nucleic acids, expression cassettes, and expression vectors encoding the antibodies and drug-peptide-binding fragments. The antibodies and binding fragments are useful for diagnosis and treatment of cancers that present a neoantigen formed by binding of an inhibitor to an oncoprotein.

### BRIEF DESCRIPTION OF THE DRAWINGS

FIG. 1A-1D show that K-Ras (G12C)-derived peptides covalently modified by the investigational inhibitor ARS1620 form functional complexes with MHC Class I heavy chain and β2-microglobulin. FIG. 1A shows that conjugate addition from the acquired cysteine (Cys12) on K-Ras (G12C) to the acrylamide group in ARS1620 yields a covalent ARS1620-K-Ras (G12C) adduct. FIG. 1B shows that ARS1620-modified peptides form functional complexes with MHC Class I heavy chain and β2-microglobulin. Recombinant MHC-I complexes were prepared by refolding of the indicated heavy chain in the presence of β2-microglobulin and the indicated peptide. For sandwich ELISA detection, the complexes were captured by the conformation-specific MHC Class I heavy chain antibody W6/32 and detected by a β2-microglobulin-specific antibody (BBM.1) (One-way ANOVA with Dunnett's correction for multiple comparisons; ns, not significant; ****, p<0.0001). Individual data points are shown with mean±standard deviation indicated. See, Table 1-1 for the SEQ ID NOs of the peptides tested. FIG. 1C shows that ARS1620-modified peptides stabilize MHC Class I on the surface of the TAP-deficient cell line T2. FIG. 1D shows thermal stability of HLA-A*02:01 MHC-I complexes loaded with ARS1620-modified peptides, which is measurable by differential scanning fluorimetry. The amino acid sequences of the peptides are set forth as

FIG. 2A-2C show characterizations of two V7-ARS A*03:01-reactive Fabs. FIG. 2A shows biolayer interferometry (BLI) sensograms of two unique Fab clones identified from phage display selection against the V7-ARS HLA-A*03:01 and K5-ARS HLA-A*02:01 MHC-I complexes and against the cognate V7-ARS peptide antigen. FIG. 2B shows differential scanning fluorimetry of Fabs in the presence of ARS1620, reduced ARS1620 (Red-ARS), the R atropoisomeric of ARS1620 (ent-ARS), or AMG-510 (Sotorasib), a structurally similar inhibitor. Data are presented as the mean±standard deviation of four replicates. FIG. 2C shows binding affinities and melting temperatures derived from FIG. 2A-2B.

FIG. 3A-3D show characterizations of six V7-AMG510 A*03:01-reactive Fabs. FIG. 3A shows biolayer interferometry (BLI) sensograms of six unique Fab clones against the target V7-AMG510 HLA-A*03:01 MHC-I complex, the cognate V7-AMG510 peptide alone, and the cognate V7 WT HLA-A*03:01 MHC I complex. FIG. 3B shows affinities of identified clones derived from FIG. 3A. All clones show no significant binding to the V7 WT HLA-A*03:01 MHC I complex up to 1 μM. Approximate IC₅₀ values for free drug (AMG510) competition for each clone are reported, as derived from FIG. 3C. FIG. 3C shows that free AMGS10 competes for binding to V7-AMG510 HLA-A*03:01 MHC I complexes to varying degrees for each identified clone. Recombinant V7-AMG510 HLA-A*03:01 was captured via streptavidin in an ELISA and increasing amounts of free AMG510 preincubated with the indicated clones in Fab format before detection of Fab binding with an anti-myc secondary IgG-HRP conjugate. Data are presented as the mean #standard deviation of three replicates. FIG. 3D shows differential scanning fluorimetry of the V7-AMGS10 HLA-A*03:01 MHC I complex with derived melting temperature. Data are presented as the mean±standard deviation of four replicates.

FIG. 4A-B show that P2B2 binds the HLA-A*03:01/V7-ARS complex with greater specificity than does P1A4. FIG. 4A shows data from a sandwich ELISA of recombinant MHC-I complexes prepared by refolding of A*03:01 in the presence of β2-microglobulin and V7-ARS. The complexes were captured by the conformation-specific antibody W6/32 and detected by the ARS1620-specific antibody (P1A4) in the presence of various amounts of free ARS1620. Data is presented as mean±standard deviation for four replicates. FIG. 4B shows data from a sandwich ELISA of recombinant MHC-I complexes prepared by refolding of HLA-A*03:01 in the presence of β2-microglobulin and V7-ARS. The complexes were captured by the conformation-specific antibody W6/32 and detected by the V7-ARS-HLA-A*03:01-specific antibody P2B2 in the presence of various amounts of free ARS1620. Data shown in FIG. 4A-4B are presented as mean±standard deviation for four replicates

FIG. 5A is a schematic representation of the dosing schema for the study designed to evaluate the impact of sotorasib treatment on P1B7 biodistribution in vivo. Tumor bearing mice (n=4/arm) were treated with vehicle or sotorasib at 100 mg/kg via oral gavage daily. Eighteen hours after the first dose, the mice received an intravenous injection of ⁸⁹Zr—P1B7 IgG. PET/CT studies were conducted at various time points post injection of the radiotracer. FIG. 5B are representative coronal PET/CT images acquired at 48 hours post injection, which show the biodistribution of ⁸⁹Zr—P1B7 in male nu/nu mice bearing either UMUC3 (KRAS G12C human bladder cancer) or H358 (KRAS G12C human non-small cell lung cancer) subcutaneous xenografts. The arrow shows the location of the flank tumor. FIG. 5C shows the relative uptake of ⁸⁹Zr—P1B7 IgG in UMUC3 or H358 tumors at 48 hours post injection. Sotorasib treatment significantly increases tumor uptake of ⁸⁹Zr—P1B7. FIG. 5D are tumor time activity curves showing the uptake of ⁸⁹Zr—P1B7 IgG in UMUC3 xenografts over time. The uptake was significantly higher in sotorasib-treated tumors compared to control at late time points post injection. FIG. 5E shows the relative uptake of ⁸⁹Zr—P1B7 IgG in tumor versus normal tissues in mice bearing UMUC3 xenografts as determined from post-mortem biodistribution studies. No significant change in radiotracer biodistribution was observed among normal organs in the vehicle versus sotorasib cohort. In contrast, radiotracer uptake was significantly higher in the tumors of mice treated with sotorasib as compared to vehicle-treated mice. All data shown in FIG. 5 C-5E are presented as mean±standard deviation

FIG. 6A is a schema showing the generalized treatment arms and dosing schedule for antitumor assessment studies comparing the effects of vehicle, sotorasib, radiolabeled P1B7 IgG, and the combination therapy. Mice received sotorasib at 30 mg/kg via oral gavage daily for 8 days. Eighteen hours after the first dose, mice received ¹⁷⁷Lu— or ²²⁵Ac—P1B7 IgG intravenously. In some cases, a second dose of radiotherapy was administered on day 7. FIG. 6B shows the fold change in tumor volume over time among male nu/nu mice bearing UMUC3 xenografts. Mice (n=8/arm) were treated with vehicle, sotorasib alone, ¹⁷⁷Lu—P1B7 IgG (˜650 mCi on day 0 and day 7), or sotorasib with ¹⁷⁷Lu—P1B7. Only combination therapy significantly delayed tumor growth. FIG. 6C shows the fold change in tumor volume on day 11, the final day on which all mice in the study were still viable. Combination therapy significantly inhibited tumor growth compared to vehicle or monotherapies. *P<0.01. FIG. 6D is a plot of mouse body weights over time. All data shown in FIG. 6B-6D are presented as mean±standard deviation.

FIG. 7A is a spider plot showing the individual fold changes in tumor volume for mice treated with (1) vehicle, (2) sotorasib, 30 mg/kg, daily oral gavage, (3) ²²⁵Ac—P1B7, single dose on day 1 at one μCi/mouse, or (4) combination sotorasib and ²²⁵Ac—P1B7. FIG. 7B is a plot showing the fold changes in tumor volumes from each treatment arm in the cohort at day 14. Combination therapy significantly retarded tumor growth compared to monotherapy and vehicle. *P<0.01. FIG. 7C is a Kaplan Meier plot showing the survival advantage associated with combination sotorasib and ²²⁵Ac—P1B7. Mice in the combination treatment arm lived significantly longer than mice in the vehicle or monotherapy arm. The endpoints were (1) weight loss >20% or (2) tumor volume>2000 mm³. *P<0.001. FIG. 7D is a plot of mouse body weights over time. Only one mouse in the ²²⁵Ac—P1B7 monotherapy arm experienced an unsafe weight loss. Data shown in FIG. 7B and FIG. 7D are presented as mean±standard deviation.

FIG. 8 shows H2122 cells that were treated either with Sotorasib (1 μM) or DMSO for 48 hours before formalin fixing and subsequent staining with PIES, a Sotorasib/MHC class I-specific antibody. Antigen retrieval was conducted using the Ventana Discovery Ultra platform using citrate buffer (pH 6) at 97° C. with 16 (top) or 32 minutes (bottom).

FIG. 9A-9C shows results of radio thin layer chromatography analysis of purified, radioisotope-labeled antibody. FIG. 9A shows ⁸⁹Zr—P1B7. FIG. 9B shows ¹⁷⁷Lu-DOTA-P1B7. FIG. 9C shows ²²⁵Ac-macropa-P1B7. All three complexes exhibited >99% radiochemical purity. Under the test conditions (20 mM citric acid), free ⁸⁹Zr/¹⁷⁷Lu/²²⁵Ac moves with solvent front, while bound ⁸⁹Zr/¹⁷⁷Lu/²²⁵Ac stays at the origin.

### DETAILED DESCRIPTION

The present disclosure relates generally to antibodies reactive with drug-peptide conjugates, as well as drug-peptide-binding fragments thereof. The present disclosure also relates to nucleic acids, expression cassettes, and expression vectors encoding the antibodies and drug-peptide-binding fragments. The antibodies and binding fragments are useful for diagnosis and treatment of cancers that express a neoantigen formed by binding of an inhibitor to an oncoprotein.

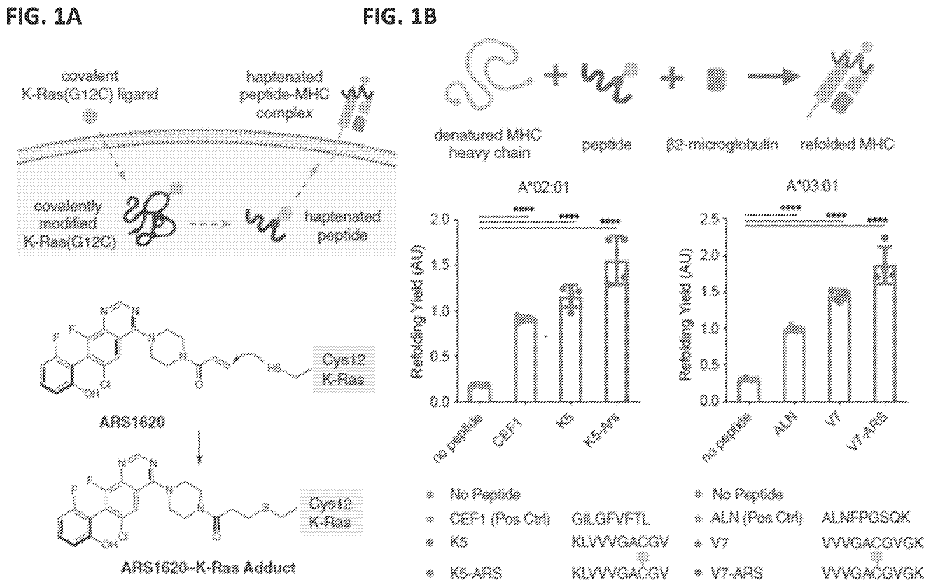

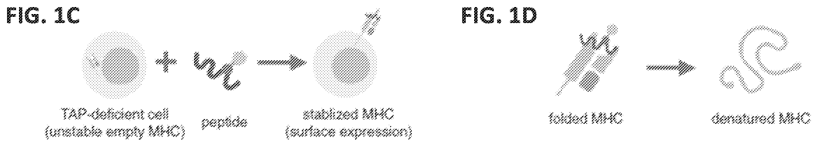

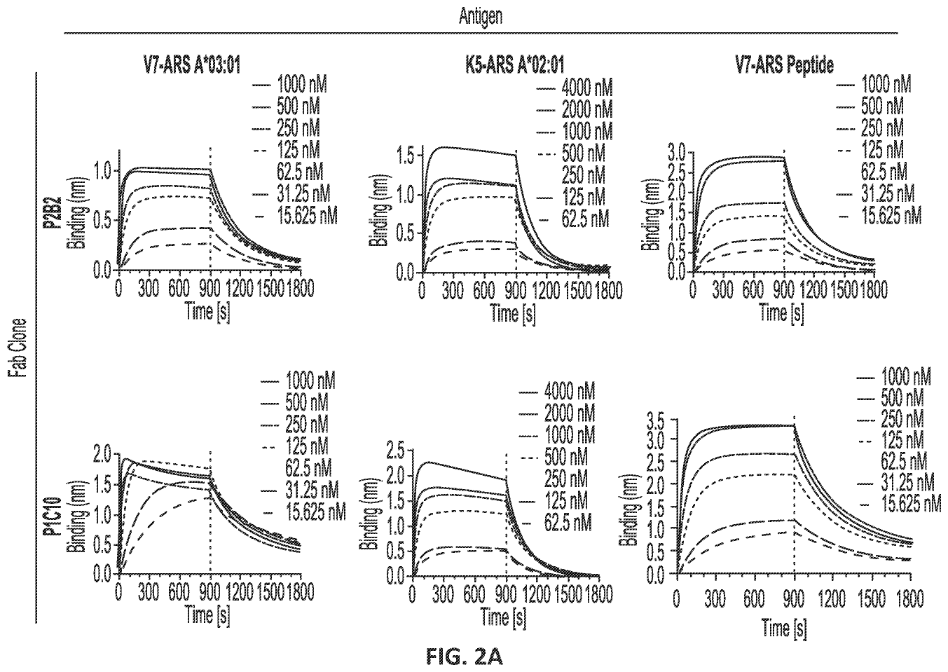

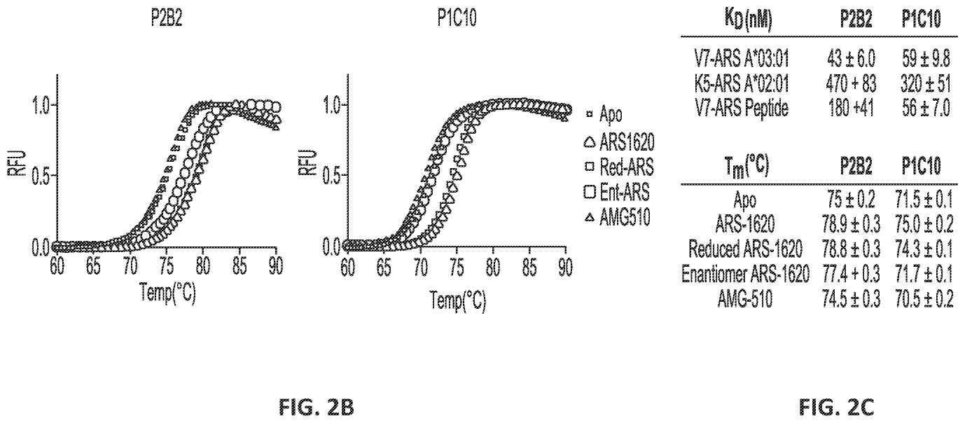

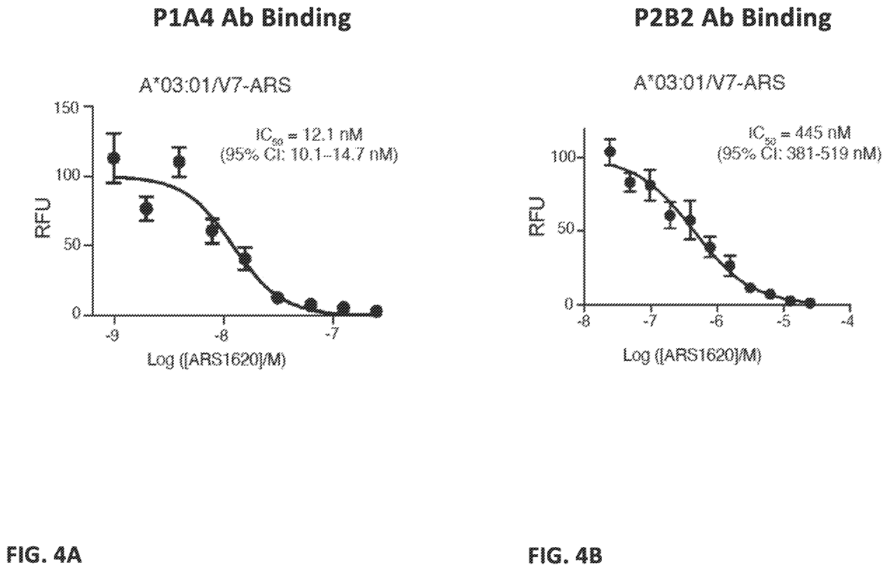

Antibody-based therapies have emerged as a powerful strategy for the management of many diverse cancers, but novel tumor-specific antigens remain challenging to identify and target. Recently, it has been established that inhibitor-modified peptide adducts derived from KRas G12C are competent for antigen presentation via MHC class I and can be targeted by antibody-based therapeutics, theoretically offering a means to directly target an intracellular oncoprotein at the cell surface (FIG. 1A). Specifically, peptides modified with ARS1620, a preclinical KRas G12C inhibitor, have been found to be competent for antigen presentation in MHC class I (FIG. 1B). First generation antibodies were isolated against ARS1620 directly, resulting in antibodies with high affinity to ARS1620 alone or when presented in MHC class I complexes (Zhang et al., Cancer Cell, 40:1060-1069, 2022). Due to this binding profile, they suffered from free drug competition in animal models and their binding sites were saturated by free drug in circulation, precluding them from specifically binding to target MHC class I complexes on the tumor. Second generation antibodies were isolated for their binding against the full MHC class I complex, resulting in antibodies that preferentially bind to ARS1620/MHC class I conjugates as compared to other ARS1620-labeled antigens (FIG. 2A-2C). One of these antibodies, P2B2, was over 37-fold more resistant to free drug competition compared to P1A4 (FIG. 4A-4B).

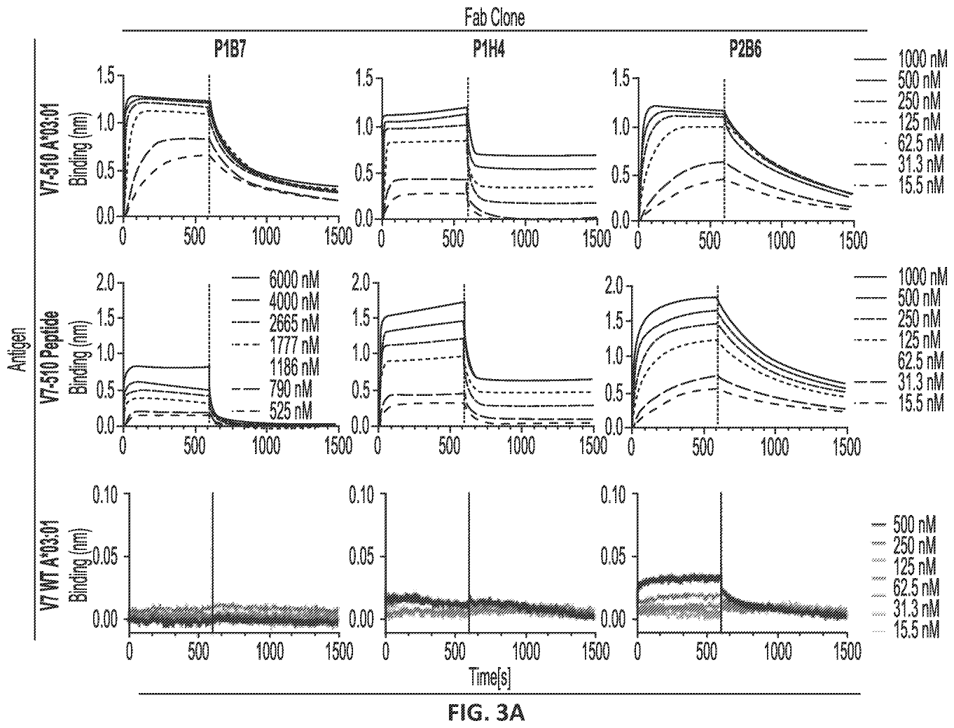

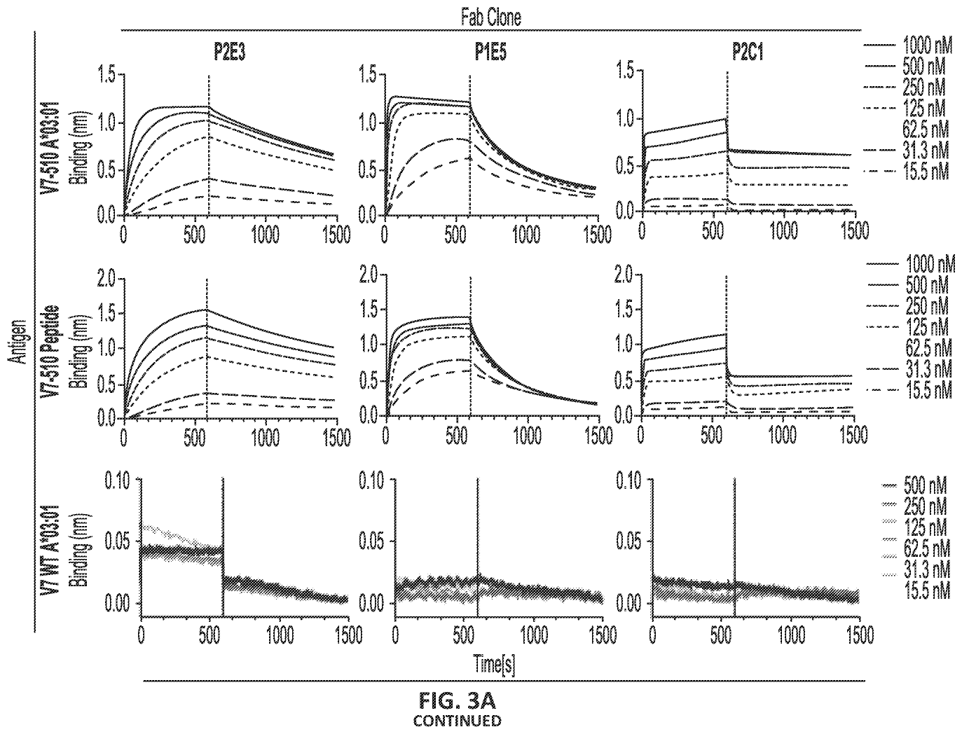

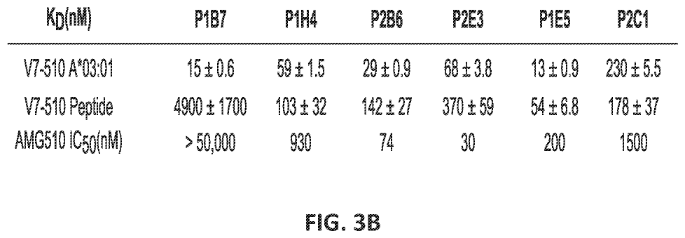

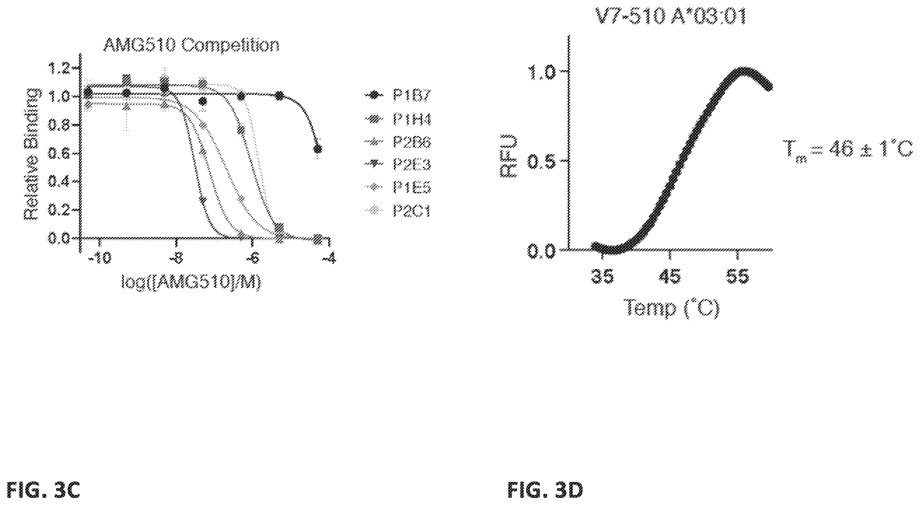

Once Sotorasib (formerly AMG510) was publicly disclosed as a clinical candidate for covalent inhibition of KRas G12C, our efforts shifted to working with this clinically relevant inhibitor. Using the approach developed for the second generation antibodies mentioned above, antibodies that bound to Sotorasib-labeled MHC class I complexes were identified (FIG. 3A-3B). The newly-identified antibodies were specific for the MHC I context, although each clone offers a unique binding profile. In particular, some antibodies are more drug specific (P1E5), while others are highly specific for the MHC I context (P1B7). The diversity of binding profiles of these unique clones offers the potential that each clone may be best suited for a specific translational application (e.g., P1B7 for radioligand therapy and P1E5 for immunohistochemical staining). Like the second generation antibodies, these antibodies demonstrate strong tolerance of free drug, with P1B7 being able to tolerate as much as 5 μM of free Sotorasib without any reduction in binding to its target MHC I complex (FIG. 3C). Additionally, the Sotorasib-labeled V7-510 A*03:01 MHC I complex was found to be stable (Tₘ=46° C.) and accommodates the Sotorasib scar well (FIG. 3D).

Expanding upon previous work (Zhang et al., Cancer Cell, 40:1060-1069, 2022; and Hattori et al., Cancer Discovery, 13:132-145, 2023), the first in vivo proof of efficacy of a therapy targeting a Sotorasib-labeled MHC I neoantigen is described in Example 4. There are currently two FDA-approved KRas G12C covalent inhibitors (Sotorasib, Adagrasib) and many others undergoing clinical trials. Existing data from the clinical use of these inhibitors indicates clear patient benefit but a relatively high de novo rate of resistance (˜50-60%) and a relatively short duration of response (˜6 months). The present disclosure describes a novel, combination therapy approach that could offer a synergistic opportunity to maximize the clinical benefit of KRas G12C covalent inhibitors for patients. In addition, the present disclosure demonstrates the ability to detect these target antigens in formalin-fixed tissue, offering the potential for an upstream prognostic immunohistochemistry application to predict efficacy of the Sotorasib-targeting antibody-based therapeutics. The data presented herein further validates inhibitor-modified peptide adducts as targets for therapeutic development and provides a potential avenue for the development of combination therapies, which is contemplated to provide superior efficacy to KRas G12C inhibitor monotherapies in patients.

### Definitions

To facilitate an understanding of the embodiments disclosed herein, a number of terms and phrases are defined below. Terms and abbreviations not defined should be accorded their ordinary meaning as used in the art.

As used herein and in the appended claims, the singular forms “a,” “an,” and “the” include plural references unless indicated otherwise. For example, “a” monocyte includes one or more monocytes.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 20 amino acids refers to 18 amino acids to 22 amino acids and includes 20 amino acids).

Numerical ranges are inclusive of the numbers defining the range (e.g., 18 to 22 amino acids encompasses 18, 19, 20, 21 and 22 amino acids).

The term “plurality” as used herein in reference to an object refers to three or more objects. For instance, “a plurality of multimers” refers to three or more multimers, preferably 3, 4, 5, 6, 7, 8, 9, 10, 100, 1,000, 10,000, 100,000, 1,000,000 or more multimers.

As used interchangeably herein, the terms “isolated” and “purified” refer to a material that is removed from at least one component with which it is naturally associated (e.g., removed from its original environment). The term “isolated,” when used in reference to a recombinant protein, refers to a protein that has been removed from the culture medium of the host cell that produced the protein. In some embodiments, an isolated protein (e.g., recombinant antibody) is at least 75%, 90%, 95%, 96%, 97%, 98% or 99% pure as determined by HPLC . . .

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. In the context of the present disclosure, the term “antigen” typically refers to a polypeptide or protein antigen at least eight amino acid residues in length, which may comprise one or more post-translational modifications.

The term “neoantigen” refers to a newly formed antigen. In the context of the present disclosure, the term “neoantigen” typically refers to an antigen that is formed upon binding of a small molecule, covalent inhibitor to an oncoprotein of a mammalian subject.

In the present disclosure, the terms “individual” and “subject” refer to a mammal. In some embodiments, the subject is a human subject, such as a cancer patient or a patient suspected of having cancer.

An “effective amount” of an agent disclosed herein is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., neoantigen-reactive antibody) effective to “treat” a disease or disorder in a subject (e.g., a mammal such as a human). An “effective amount” or an “amount sufficient” of an agent or agents may be administered in one or more doses.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival of an individual not receiving treatment. “Palliating” a disease or disorder means that the extent and/or undesirable clinical manifestations of the disease or disorder are lessened and/or time course of progression of the disease or disorder is slowed, as compared to the expected untreated outcome. Further, palliation and treatment do not necessarily occur by administration of one dose, but often occur upon administration of a series of doses.

“Treating” cancer means to bring about a beneficial clinical result such as causing remission or otherwise prolonging survival as compared to expected survival in the absence of treatment. In some embodiments, “treating” cancer comprises shrinking the size of a tumor or otherwise reducing viable cancer cell numbers. In other embodiments, “treating” cancer comprises delaying growth of a tumor.

As used herein, a “Fab” refers to an antigen binding fragment of an antibody. As used herein, a “F(ab′);” refers to a fragment of an antibody consisting of two Fabs bound by the hinge region of the antibody, such as is produced by the digestion of an antibody by the enzyme pepsin, for example. As used herein, a “Fv” refers to a fragment of an antibody that contains the variable heavy and variable light chains. As used herein, an “Sfv” refers to a single chain Fv, an Fv that has a peptide linker between the variable heavy and variable light domains.

As used herein, a “bispecific T-cell engager” (“BiTE”) refers to a single peptide chain that contains two Sfvs from two different antibodies, where one Sfv is binds to a cancer cell, and the second Sfv binds to T cells via the CD3 receptor.

As used herein, a “chimeric antigen receptor” (“CAR”) refers to an engineered receptor that has both antigen-binding function and T-cell activating function. CARs may be specific to proteins selectively expressed on the surfaces of cancer cells.

As used herein, a “bispecific antibody” refers to an antibody, typically an engineered antibody, which contains two different specific antigen binding elements. As used herein, a “trispecific antibody” refers to an antibody, typically an engineered antibody, which contains three different specific antigen binding elements.

The terms “percent (%) amino acid sequence identity” and “percent identity” and “sequence identity” when used with respect to an amino acid sequence (reference polypeptide sequence) is defined as the percentage of amino acid residues in a candidate sequence (e.g., the subject antigen) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into an antigen of interest and the products screened for a desired activity, e.g., increased stability and/or immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

Conservative amino acid substitutions will involve exchanging a member of one of these classes with another member of the same class. Non-conservative amino acid substitutions will involve exchanging a member of one of these classes with a member of another class.

As used herein, the term “excipient” refers to a compound present in a composition comprising an active ingredient (e.g., ARS1620, AMG510, antibody, antibody fragment, BITE, CAR, etc.). Pharmaceutically acceptable excipients are inert pharmaceutical compounds, and may include for instance, solvents, bulking agents, buffering agents, tonicity adjusting agents, and preservatives (Pramanick et al., Pharma Times, 45:65-77, 2013). In some embodiments the compositions of the present disclosure comprise an excipient that functions as one or more of a solvent, a bulking agent, a buffering agent, and a tonicity adjusting agent (e.g., sodium chloride in saline may serve as both an aqueous vehicle and a tonicity adjusting agent).

### 1. Identification of Neoantigen-Reactive Antibodies

The present disclosure details a powerful approach, referred to herein as “Chemically Controlled monoclonal Antibody Target Engagement”, or “CheCmATE” by using tumor-specific chemical reactivities to create neoantigens that can be recognized in pMHCs without allelic restriction (FIG. 1A). This is accomplished by using an oncoprotein-specific hapten that covalently reacts in a tumor-specific manner to create a neoantigen. Specific recognition of the hapten by engineered antibodies serves to direct an immune response against cancer cells presenting haptenized peptides as pMHCs on their cell surfaces. A “hapten” is a small molecular weight compound that elicits an immune response only when covalently linked to a larger “carrier” molecule such as a protein, but not on its own (Erkes et al., J Immunol Res, 2014:175265, 2014). Initially, acquired cysteines on oncoproteins were targeted for haptenization as the acquired cysteines represent chemically-reactive handles that effectively distinguish between healthy and cancerous cells (Visscher et al., Current Opinion in Chemical Biology 30, 61-67, 2016). Non-cancerous cells should not produce the neoantigen since the wild type protein lacks the cysteine required for reaction with the hapten. By engineering an antibody specific to the small molecule hapten using the CheCmATE approach instead of to a single pMHC, covalently modified peptides presented by any MHC molecule on the cell surface can be targeted as long as the hapten is reasonably solvent-exposed. In this way, MHC and even carrier peptide specificities are bypassed, resulting in reasonably selective hapten reactivity. This means that any potential proteasomal degradation product containing the modified cysteine successfully loaded into any MHC allele should form a competent epitope for targeting with the CheCmATE approach. Additionally, antibodies reactive with an oncoprotein-specific hapten could be suitable for use as a monotherapy; such antibodies are also fundamentally synergistic with irreversible inhibitors. This means that oncoprotein-specific, irreversible inhibitors that already exist or are in development can be repurposed with the CheCmATE approach as combination therapies or as an option in case resistance to the targeted inhibitor monotherapy.

Using pharmacological perturbation to create neo-epitopes for immuno-targeting represents a new modality that combines the strength of small molecule- and protein-based therapeutics. The CheCmATE approach has numerous advantages. 1) Oncogenic drivers are directly targeted as they are intrinsically tumor-specific and required for tumor maintenance. 2) Though the tumor-specific antigen is intracellular, antigen presentation brings peptide fragments to the cell surface. 3) Inhibiting the function of the tumor-specific antigen is not necessary since the small molecule serves primarily as a haptenizing agent. 4) Because the approach relies on the existence rather than inhibition of a tumor-specific antigen, it can retain efficacy even in the case of resistance to the small molecule as long as it is still able to covalently bind the target. 5) Full target engagement is unnecessary since substoichiometric target engagement can be sufficient to generate hapten-pMHCs. 6) As the immunotherapy effect is hapten-mediated, the relatively acute exposure provided by the orally bioavailable small molecule hapten allows tighter temporal control over the potentially damaging side effects common to immunotherapies, which are typically affected by long-lived species like antibodies and engineered cells.

The CheCmATE approach was further tested in an exemplary model system involving a commonly mutated oncogene having a mutation resulting in an acquired cysteine, namely KRas G12C. Antibodies specific to the preclinical KRas G12C inhibitor ARS-1620 and the FDA-approved drug AMG-510 were identified and characterized. Structures of exemplary targeted covalent inhibitors of KRas G12C are shown in Table I.

ARS-1620 shows high specificity for KRas G12C over other cellular proteins and shows no appreciable binding to wild type KRas lacking the acquired cysteine (Visscher et al., Current Opinion in Chemical Biology, 30:61-67, 2016). One antibody, P1A4, was found to detect ARS-1620 modified peptides in pMHCs on the cell surface of KRas G12C cell lines treated with ARS-1620 and the MHC presentation of ARS-1620 was found to be sufficient to recruit a cytotoxic T-cell response through a bispecific T-cell engager (BiTE) incorporating the ARS-1620-binding fragment of P1A4. P1A4 and four other anti-ARS1620 Fabs were previously isolated and characterized (see, WO 2021/014417 of Craik et al.). P1A4 was found to bind free ARS-1620 with high affinity, which may be less desirable for use as a immunotherapeutic agent. As described in Example 1, two further antibodies, P2B2 and P1C10 having high affinity for the KRas-ARS-1620 neoantigen were identified during development of the present disclosure. The two new antibodies have lower affinities for free ARS-1620 than P1A4, and therefore represent an improvement over the original anti-ARS1620 antibodies.

AMG510 also shows high specificity for KRas G12C and binds irreversibly (Canon et al., Nature, 575:217-223, 2019; and Fakih et al., J Clin Oncol, 37:3003, 2019). AMG510, also known as sotorasib, has recently been approved by the FDA for treatment of adult patients with KRAS G12C-mutated, locally advanced or metastatic non-small cell lung cancer. AMG510 (sotorasib) is marketed as LUMAKRAS™ by Amgen Inc. (Thousand Oaks, CA) in the United States. AMG510 has been reported to rapidly engage cellular KRas G12C proteins and drive tumor regression in mouse models and clinically (Canon et al., 2019; Fakih et al., 2019). However, clinical resistance to AMG510 has already been observed (Awad et al., 2021; Koga et al., 2021; Tanaka et al., 2021), with various mechanisms including mutations on the WT KRas allele in trans. However, most resistant tumors retain the expression of KRas G12C (Awad et al., 2021). CheCmATE will therefore still generate neoantigens from the KRas G12C protein, regardless of the mutation status of the other allele, and thus the cancer cells can still be targeted. Six antibodies, P1B7, P1H4, P2B6, P2E3, P1E5, and P2C1 were identified as having high affinities for the KRas-AMG-510 neoantigen and are therefore promising therapeutic agents.

The antibody or antigen-binding fragment of the present disclosure binds to an antigen formed by binding of an inhibitor to a protein, such as a neoantigen formed by binding of in inhibitor to an oncoprotein. In some preferred embodiments, the antibody or antigen binding fragment does not bind or binds with lower affinity to a control antigen comprising the same sequence as the protein but devoid of the inhibitor (e.g., oncoprotein). In some embodiments, the neoantigen is presented on a cell (e.g., cancer cell) surface by a class I molecule (heavy chain and beta-2-microglobulin) as a peptide major histocompatibility complex I (pMHC-I), and/or the neoantigen is presented on a cell (e.g., antigen presenting cell) surface by a class II molecule (alpha chain and beta chain) as a peptide major histocompatibility complex II (pMHC-II). In some embodiments, the antibody or antigen binding fragment binds to pMHC-I and/or pMHC-II in an MHC allele-agnostic manner (e.g., capable of binding pMHCs formed from a plurality of MHC alleles). In some embodiments, the antibody or antigen binding fragment binds to pMHC-I and/or pMHC-II in an MHC allele-specific manner (e.g., capable of preferentially binding pMHCs formed one or more MHC-I alleles). For instance, in some embodiments, the antibody or antigen binding fragment binds with a higher affinity to a pMHC comprising HLA-A*03:01 than a pMHC comprising HLA-A*02:01.

TABLE I
Exemplary KRas G12C Targeted Covalent Inhibitors

| Compound | Structure |
|---|---|
| ARS-1620 (ARS1620) | 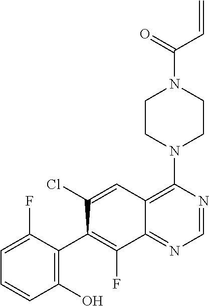 |
| reduced ARS-1620 analogue | 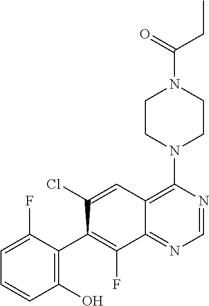 |
| AMG-500 (AMG510) Sotorasib | 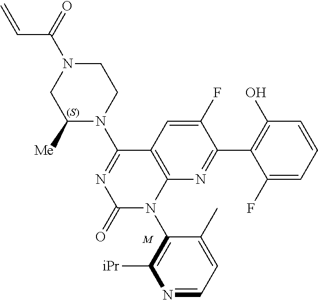 |
| | CAS Registry No. 2252403-56-6 |

Using a hapten on the outside of a cancer cell has been demonstrated to recruit an anti-cancer immune response (Rullo et al., Angewandte Chemie International Edition 55, 3642-3646, 2016), and covalently modified peptides derived from intracellular proteins have been shown to be presented in pMHCs (Padovan et al., Eur J Immunol, 27:1303-1307, 1997; and Martin and Weltzien, Int Arch Allergy Immunol, 104:10-16, 1994). However, the present disclosure shows that a haptenized pMHC can be directly targeted as an immunotherapy approach against intracellular oncoproteins. Moreover, the CheCmATE approach is the first approach to target pMHCs of intracellular antigens in a completely MHC allele and peptide (register/orientation) agnostic way.

The present disclosure provides multiple new antibodies that can be used with the CheCmATE approach in order to treat various cancers. Six of the antibodies can be used in combination with AMG-510. Two other antibodies can be used in combination with the ARS-1620. In some embodiments, the antibody or antibody fragment binds to Sotorasib-MHC I, and comprises the amino acid sequences of the light chain and heavy chain CDRs of P1B7, P1H4, P2B6, P2E3, P1E5 or P2C1. In some embodiments, the antibody or antibody fragment binds to Sotorasib-MHC I, and comprises the amino acid sequences of the light chain and heavy chain CDRs of P1B7. In some embodiments, the antibody or antibody fragment binds to ARS1620-MHC I, and comprises the amino acid sequences of the light chain and heavy chain CDRs of P1A4, P2B2 or P1C10.

In some embodiments, the antibody or antibody fragment binds to Sotorasib-MHC I, and comprises the amino acid sequences of the heavy chain variable domain (VH) and the light chain variable domain (VL) of P1B7, P1H4, P2B6, P2E3, P1E5 or P2C1. In some embodiments, the antibody or antibody fragment binds to Sotorasib-MHC I, and comprises the amino acid sequences of the VH and the VL of P1B7. In some embodiments, the antibody or antibody fragment binds to ARS1620-MHC I, and comprises the amino acid sequences of the VH and the VL of P1A4, P2B2 or P1C10. In some embodiments, the antibody is an IgG1 antibody.

Further provided are immunoconjugates of these antibodies, as well as nucleic acids encoding the antibodies, expression cassettes comprising the nucleic acids, and host cells comprising the expression cassettes. The present disclosure also includes a method for production of recombinant antibodies, methods of characterizing biopsy samples from patients, methods for treating cancer, and compositions for use in the treatment of cancer, utilizing the provided antibodies or fragments thereof.

### 2. Antibody Conjugates

The disclosure also provides antibodies or antibody fragments that are conjugated to one or more detectable markers (also referred to herein as detectable labels) or cytotoxic agents. The conjugates need not be composed of an entire, intact antibody and instead can be composed of any one of the antibody fragments disclosed herein.

In certain embodiments, the antibodies or antibody fragments thereof provided herein are labeled such that the antibodies or antibody fragments can be detected, e.g., once bound to drug-peptide complex (e.g., inhibitor-MHC I). The labels may be conjugated directed to the antibody or antibody fragment, or the label may be attached to the antibody or antibody fragment via a linker moiety. Labels for antibodies are well known in the art and include but are not limited to biotin, fluorescent dyes, fluorescent proteins and enzymes. Labels include, but are not limited to, directly detected labels (e.g., fluorescent, chromophoric, electron-dense, chemiluminescent, and radioactive labels), as well as labels such as enzymes or ligands that are indirectly detected, e.g., through an enzymatic reaction or molecular interaction. Exemplary labels include, but are not limited to, radioisotopes, fluorophores such as rare earth chelates or fluorescein and its derivatives, rhodamine and its derivatives, dansyl, umbelliferone, luceriferases, luciferin, 2,3-dihydrophthalazinediones, horseradish peroxidase, alkaline phosphatase, β-galactosidase, glucoamylase, lysozyme, saccharide oxidases, heterocyclic oxidases in combination with an enzyme that employs hydrogen peroxide to oxidize a dye precursor such as horseradish peroxidase, lactoperoxidase, or microperoxidase, biotin/avidin, spin labels, bacteriophage labels, stable free radicals, and similar molecules . . .

In some embodiments, the disclosure provides antibodies or antibody fragments that are labeled with a radionuclide (also referred to herein as a radioisotope). In some embodiments, the disclosure provides antibody-radionuclide conjugates (ARCs). In some embodiments, the radioisotope-labeled antibody or antibody fragment has the formula: Radioisotope-Linker-Antibody (R*-L-Ab), wherein R* is a radioisotope, L is a linker, and Ab is an antibody or antibody fragment that specifically binds a drug-peptide conjugate, such as ARS1620-MHC I or Sotorasib-MHC I. In some embodiments, the linker is a macrocyclic chelator. In an exemplary embodiment, the macrocyclic chelator comprises tetraxetan. In some embodiments, the radioisotope is a therapeutic radioisotope. In other embodiments, the radioisotope is a diagnostic radioisotope.

As used herein, the term “therapeutic radioisotope” refers to an alpha-emitter and/or a beta-minus-emitter, preferably with a half-life of hours to days. In some embodiments, the therapeutic radioisotope is selected from the group consisting of ⁶⁷Cu, ⁹⁰Y, ¹³¹I, ¹⁵³Sm, ¹⁷⁷Lu, ²¹¹At, ²¹²Pb, ²²³Ra, ²²⁵Ac, and ²²⁷Th. In an exemplary embodiment, the therapeutic radioisotope is ¹⁷⁷Lu.

The term “diagnostic radioisotope” as used herein, refers to a positron-emitter, preferably with a half-life of minutes to hours. In some embodiments, the diagnostic radioisotope is selected from the group consisting of ¹⁸F, ⁴⁴Sc ⁶⁴Cu, ⁶⁸Ga, ⁸⁹Zr, and ¹²⁴I. In an exemplary embodiment, the diagnostic radioisotope is ⁸⁹Zr.

In one specific embodiment, the disclosure provides antibody-drug conjugates (ADCs). As used herein, a cytotoxic agent (drug) is a compound that, depending on the dosage required, generally interferes with or inhibits cell growth, or kills cells to which the cytotoxic agent is administered. Examples of classes of compounds that may be used as cytotoxic agents in the ADCs of the present disclosure include but are not limited to kinase inhibitors, cytoskeletal disruptors, anthracyclines such as daunomycin or doxorubicin, epothilones, Type I topoisomerase inhibitors, Type II topoisomerase inhibitors, histone deacetylase inhibitors, nucleotide analogs and precursor analogs, alkylating agents, platinum-based agents, Vinca alkaloids and derivatives, calicheamycins, and retinoids. Other examples of cytotoxic agents to which the antibody or antibody fragment may be linked include but are not limited to, monomethyl auristatin F (MMAF), monomethyl auristatin E (MMAE), duocarmycin, maytansinoids, methotrexate, vindesine, a taxane such as docetaxel, paclitaxel, larotaxel, tesetaxel, or ortataxel, a dolastatin or a trichothecene.

### 3. Pharmaceutical Compositions

Further provided are pharmaceutical compositions comprising the antibodies or antibody fragments described. The pharmaceutical compositions comprise at least one antibody or antibody fragment of the present disclosure and a pharmaceutical excipient. In some embodiments, the antibody or antibody fragment is radiolabeled. The pharmaceutical compositions may be administered, or may be formulated to be administered, by parenteral administration, for example, intravenous administration. Intravenous administration may be done by injection or infusion.

The present compositions will contain a therapeutically effective amount of the antibodies or antibody fragments, together with a suitable amount of a pharmaceutically acceptable vehicle so as to provide the form for proper administration to a patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the disclosure is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the disclosure and pharmaceutically acceptable vehicles should be sterile. Water is one example of a vehicle when the compound of the disclosure is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The pharmaceutical compositions may further contain one or more auxiliary substance, such as wetting or emulsifying agents, pH buffering agents, or adjuvants to enhance the effectiveness thereof.

In another embodiment, the compounds and/or compositions of the disclosure (antibodies) are formulated in accordance with routine procedures as a pharmaceutical composition adapted for intravenous administration to mammals, including humans. Typically, compounds and/or compositions of the disclosure for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the disclosure is to be administered by infusion, it can be dispensed, for example, with an infusion bottle containing sterile pharmaceutical grade water or saline. Where the compound of the disclosure is administered by injection, an ampoule of sterile water for injection or saline can be provided so that the ingredients may be mixed prior to administration.

The amount of a compound of the disclosure that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. In specific embodiments of the disclosure, the oral dose of at least one compound of the present disclosure is about 0.01 milligram to about 100 milligrams per kilogram body weight, or from about 0.1 milligram to about 50 milligrams per kilogram body weight, or from about 0.5 milligram to about 20 milligrams per kilogram body weight, or from about 1 milligram to about 10 milligrams per kilogram body weight.

Suitable dosage ranges for parenteral, for example, intravenous (IV) administration are 0.01 milligram to 100 milligrams antibody or antibody fragment per kilogram body weight, 0.1 milligram to 35 milligrams per kilogram body weight, and I milligram to 10 milligrams per kilogram body weight. In other embodiments, a composition of the disclosure for parenteral, for example, intravenous administration includes about 0.001 milligram to about 2000 milligrams of a compound of the disclosure, from about 0.01 milligram to about 1000 milligrams of a compound of the disclosure, from about 0.1 milligram to about 500 milligrams of a compound of the disclosure, or from about 1 milligram to about 200 milligrams of a compound of the disclosure.

The disclosure also provides pharmaceutical packs or kits comprising one or more containers filled with one or more of the radiolabeled antibodies or antibody fragments of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

A therapeutically effective amount of the antibodies or antibody fragments in the composition should be administered, in which “a therapeutically effective amount” is defined as an amount that is sufficient to produce a desired prophylactic, therapeutic or ameliorative response in a subject. The amount needed will vary depending upon the antibodies or antibody fragments used and the species and weight of the subject to be administered, but may be ascertained using standard techniques.

### Enumerated Embodiments

1. An antibody or antigen binding fragment thereof, wherein the antibody or fragment binds to an antigen comprising the amino acid sequence VVVGAC (510) GVGK (SEQ ID NO: 100), wherein 510 is AMG-510 (sotorasib) and the antibody or fragment comprises:
(a) a light chain variable region comprising a CDRL1 of SEQ ID NO:49, a CDRL2 of SEQ ID NO:50, and a CDRL3 of SEQ ID NO:51, and a heavy chain variable region comprising a CDRH1 of SEQ ID NO:52, a CDRH2 of SEQ ID NO:53, and a CDRH3 of SEQ ID NO:54;
(b) a light chain variable region comprising a CDRL1 of SEQ ID NO:57, a CDRL2 of SEQ ID NO:58, and a CDRL3 of SEQ ID NO:59, and a heavy chain variable region comprising a CDRH1 of SEQ ID NO:60, a CDRH2 of SEQ ID NO:61, and a CDRH3 of SEQ ID NO:62;
(c) a light chain variable region comprising a CDRL1 of SEQ ID NO:65, a CDRL2 of SEQ ID NO:66, and a CDRL3 of SEQ ID NO:67, and a heavy chain variable region comprising a CDRH1 of SEQ ID NO:68, a CDRH2 of SEQ ID NO:69, and a CDRH3 of SEQ ID NO:70;
(d) a light chain variable region comprising a CDRL1 of SEQ ID NO:73, a CDRL2 of SEQ ID NO:74, and a CDRL3 of SEQ ID NO:75, and a heavy chain variable region comprising a CDRH1 of SEQ ID NO:76, a CDRH2 of SEQ ID NO:77, and a CDRH3 of SEQ ID NO:78;
(e) a light chain variable region comprising a CDRL1 of SEQ ID NO:81, a CDRL2 of SEQ ID NO:82, and a CDRL3 of SEQ ID NO:83, and a heavy chain variable region comprising a CDRH1 of SEQ ID NO:84, a CDRH2 of SEQ ID NO:85, and a CDRH3 of SEQ ID NO:86; or
(f) a light chain variable region comprising a CDRL1 of SEQ ID NO:89, a CDRL2 of SEQ ID NO:90, and a CDRL3 of SEQ ID NO:91, and a heavy chain variable region comprising a CDRH1 of SEQ ID NO:92, a CDRH2 of SEQ ID NO:93, and a CDRH3 of SEQ ID NO:94,
optionally wherein the antibody or fragment does not bind or binds with lower affinity to a control antigen comprising the amino acid sequence VVVGACGVGK (SEQ ID NO:98), and/or
optionally wherein the antigen is presented on a cell (e.g., cancer cell) surface by a class I molecule (heavy chain and beta-2-microglobulin) as a peptide major histocompatibility complex I (pMHC-I).

2. The antibody or fragment of embodiment 1, wherein the antibody or fragment is human or humanized.

3. The antibody or fragment of embodiment 1, wherein:
(a) the light chain variable region comprises the amino acid sequence of SEQ ID NO:47 and the heavy chain variable region comprises the amino acid sequence of SEQ ID NO:48;
(b) the light chain variable region comprises the amino acid sequence of SEQ ID NO:55 and the heavy chain variable region comprises the amino acid sequence of SEQ ID NO:56;
(c) the light chain variable region comprises the amino acid sequence of SEQ ID NO:63 and the heavy chain variable region comprises the amino acid sequence of SEQ ID NO:64;
(d) the light chain variable region comprises the amino acid sequence of SEQ ID NO:71 and the heavy chain variable region comprises the amino acid sequence of SEQ ID NO:72;
(e) the light chain variable region comprises the amino acid sequence of SEQ ID NO:79 and the heavy chain variable region comprises the amino acid sequence of SEQ ID NO:80; or
(f) the light chain variable region comprises the amino acid sequence of SEQ ID NO:87 and the heavy chain variable region comprises the amino acid sequence of SEQ ID NO:88.

4. The antibody or fragment of embodiment 3, comprising:
(a) a light chain comprising the amino acid sequence of SEQ ID NO: 11 and a heavy chain comprising the amino acid sequence of SEQ ID NO: 17, or a light chain comprising an amino acid sequence having at least 95% sequence identity to SEQ ID NO: 11 and a heavy chain comprising an amino acid sequence having at least 95% sequence identity to SEQ ID NO:17;
(b) a light chain comprising the amino acid sequence of SEQ ID NO: 12 and a heavy chain comprising the amino acid sequence of SEQ ID NO: 18, or a light chain comprising an amino acid sequence having at least 95% sequence identity to SEQ ID NO: 12 and a heavy chain comprising an amino acid sequence having at least 95% sequence identity to SEQ ID NO:18;
(c) a light chain comprising the amino acid sequence of SEQ ID NO: 13 and a heavy chain comprising the amino acid sequence of SEQ ID NO:19, or a light chain comprising an amino acid sequence having at least 95% sequence identity to SEQ ID NO: 13 and a heavy chain comprising an amino acid sequence having at least 95% sequence identity to SEQ ID NO:19;
(d) a light chain comprising the amino acid sequence of SEQ ID NO:14 and a heavy chain comprising the amino acid sequence of SEQ ID NO:20, or a light chain comprising an amino acid sequence having at least 95% sequence identity to SEQ ID NO: 14 and a heavy chain comprising an amino acid sequence having at least 95% sequence identity to SEQ ID NO:20;
(e) a light chain comprising the amino acid sequence of SEQ ID NO: 15 and a heavy chain comprising the amino acid sequence of SEQ ID NO:21, or a light chain comprising an amino acid sequence having at least 95% sequence identity to SEQ ID NO: 15 and a heavy chain comprising an amino acid sequence having at least 95% sequence identity to SEQ ID NO:21; or
(f) a light chain comprising the amino acid sequence of SEQ ID NO:16 and a heavy chain comprising the amino acid sequence of SEQ ID NO:22, or a light chain comprising an amino acid sequence having at least 95% sequence identity to SEQ ID NO: 16 and a heavy chain comprising an amino acid sequence having at least 95% sequence identity to SEQ ID NO:22.

5. The antibody or fragment of embodiment 1, comprising:
(i) a light chain variable region comprising a CDRL1 of SEQ ID NO:49, a CDRL2 of SEQ ID NO:50, and a CDRL3 of SEQ ID NO:51, and a heavy chain variable region comprising a CDRH1 of SEQ ID NO:52, a CDRH2 of SEQ ID NO:53, and a CDRH3 of SEQ ID NO:54;
(ii) a light chain variable region comprising the amino acid sequence of SEQ ID NO:47 and a heavy chain variable region comprising the amino acid sequence of SEQ ID NO:48; or
(iii) a light chain comprising the amino acid sequence of SEQ ID NO:11 and a heavy chain comprising the amino acid sequence of SEQ ID NO: 17, or a light chain comprising an amino acid sequence having at least 95% sequence identity to SEQ ID NO: 11 and a heavy chain comprising an amino acid sequence having at least 95% sequence identity to SEQ ID NO:17.

6. The antibody or fragment of embodiment 1, wherein the antibody or fragment is a fragment, preferably wherein the fragment is selected from the group consisting of a Fab, F(ab′)₂, Fv and Sfv.

7. The antibody or fragment of embodiment 1, wherein the antibody or fragment is a full-length human immunoglobulin g (IgG) antibody, optionally wherein the IgG is an IgG1 or an IgG4.

8. The antibody or fragment of embodiment 1, wherein the antibody or fragment comprises:
(a) a bispecific antibody, optionally wherein the bispecific antibody is in a format selected from the group consisting of a bispecific IgG (BsIgG), an appended IgG, a bispecific antibody fragment, a bispecific fusion protein, and a bispecific antibody conjugate; or
(b) a trispecific antibody.

9. The antibody or fragment of embodiment 1, wherein the antibody or fragment comprises a bispecific T-cell engager (BITE).

10. The antibody or fragment of embodiment 1, wherein the antibody or fragment comprises a chimeric antigen receptor (CAR).

11. An immunoconjugate comprising the antibody or fragment of any one of embodiments 1-7 and a detectable marker or cytotoxic agent.

12. The immunoconjugate of embodiment 11, wherein the immunoconjugate comprises a detectable marker, optionally wherein the detectable marker is selected from the group consisting of a radioisotope, a metal chelator, an enzyme, a fluorescent compound, a bioluminescent compound, and a chemiluminescent compound.

13. The immunoconjugate of embodiment 11, wherein the immunoconjugate comprises a cytotoxic agent, optionally wherein the cytotoxic agent is selected from the group consisting of an alkylating agent, an antimetabolite, an mitotic inhibitor, an antineoplastic antibiotic, a radionuclide, and a toxin.

14. An isolated nucleic acid encoding the antibody or fragment of any one of embodiments 1-10.

15. An expression cassette comprising the nucleic acid of embodiment 14, in operable combination with a regulatory sequence.

16. A host cell comprising the expression cassette of embodiment 15 or an expression vector comprising the expression cassette.

17. A method for the production of a recombinant antibody or fragment thereof, the method comprising:
a) culturing the host cell of embodiment 16 under conditions suitable for expression of the antibody or fragment; and
b) recovering the antibody or fragment from the host cell or its cell culture supernatant.

18. A method for characterizing a biopsy sample from a patient, comprising:
a) contacting the sample with the antibody or fragment of any one of embodiments 1-7 or the immunoconjugate of embodiment 12; and
b) detecting the binding of the antibody or fragment to cells of the sample, wherein the patient has received an effective amount of sotorasib (AMG-510).

19. A method for treating cancer, the method comprising:
administering to a patient with cancer an effective amount of the antibody or fragment of any one of embodiments 1-10 or the immunoconjugate of embodiment 13, wherein the patient has received or is receiving an effective amount of sotorasib (AMG-510).

20. The method of embodiment 19, wherein the cancer is an advanced solid tumor, optionally wherein the advanced solid tumor is a metastatic tumor or unresectable tumor.

21. The method of embodiment 19 or 20, wherein the cancer is a carcinoma.

22. The method of embodiment 21, wherein the carcinoma is a non-small cell lung carcinoma (NSCLC), a colorectal adenocarcinoma (CRC), or a pancreatic adenocarcinoma.

23. The method of any one of embodiments 19-22, wherein a kirsten rat sarcoma virus homolog (Kras) with a G12C mutation is expressed in cells of the cancer 24. A composition for use in a method for treating cancer in a patient, comprising: an effective amount of the antibody or fragment of any one of embodiments 1-10 or the immunoconjugate of embodiment 13, wherein the patient has received or is receiving an effective amount of sotorasib (AMG-510), and a kirsten rat sarcoma virus homolog (Kras) with a G12C mutation is expressed in cells of the cancer.

25. An antibody or antigen binding fragment thereof, wherein the antibody or fragment binds to an antigen comprising the amino acid sequence GAC(1620)GVGKSAL (SEQ ID NO: 2), wherein 1620 is ARS-1620 and the antibody or fragment comprises:
(a) a light chain variable region comprising a CDRL1 of SEQ ID NO:33, a CDRL2 of SEQ ID NO:34, and a CDRL3 of SEQ ID NO:35, and a heavy chain variable region comprising a CDRH1 of SEQ ID NO:36, a CDRH2 of SEQ ID NO:37, and a CDRH3 of SEQ ID NO:38; or
(b) a light chain variable region comprising a CDRL1 of SEQ ID NO:41, a CDRL2 of SEQ ID NO:42, and a CDRL3 of SEQ ID NO:43, and a heavy chain variable region comprising a CDRH1 of SEQ ID NO:44, a CDRH2 of SEQ ID NO:45, and a CDRH3 of SEQ ID NO:46,
optionally wherein the antibody or fragment does not bind or binds with lower affinity to a control antigen comprising the amino acid sequence GACGVGKSAL (SEQ ID NO:1), and/or
optionally wherein the antigen is presented on a cell (e.g., cancer cell) surface by a class I molecule (heavy chain and beta-2-microglobulin) as a peptide major histocompatibility complex I (pMHC-I).

26. The antibody or fragment of embodiment 25, wherein the antibody or fragment is human or humanized.

27. The antibody or fragment of embodiment 25, wherein the light chain variable region comprises the amino acid sequence of SEQ ID NO:31 and the heavy chain variable region comprises the amino acid sequence of SEQ ID NO:32.

28. The antibody or fragment of embodiment 27, comprising a light chain comprising the amino acid sequence of SEQ ID NO:6 and a heavy chain comprising the amino acid sequence of SEQ ID NO:9, or a light chain comprising an amino acid sequence having at least 95% sequence identity to SEQ ID NO:6 and a heavy chain comprising an amino acid sequence having at least 95% sequence identity to SEQ ID NO:9.

29. The antibody or fragment of embodiment 25, wherein the light chain variable region comprises the amino acid sequence of SEQ ID NO:39 and the heavy chain variable region comprises the amino acid sequence of SEQ ID NO:40.

30. The antibody or fragment of embodiment 29, comprising a light chain comprising the amino acid sequence of SEQ ID NO:7 and a heavy chain comprising the amino acid sequence of SEQ ID NO:10, or a light chain comprising an amino acid sequence having at least 95% sequence identity to SEQ ID NO:7 and a heavy chain comprising an amino acid sequence having at least 95% sequence identity to SEQ ID NO:10.

31. The antibody or fragment of embodiment 25, wherein the antibody or fragment is a fragment, preferably wherein the fragment is selected from the group consisting of a Fab, F(ab′)2, Fv and Sfv.

32. The antibody or fragment of embodiment 25, wherein the antibody or fragment is a full-length human immunoglobulin g (IgG) antibody, optionally wherein the IgG is an IgG1 or an IgG4.

33. The antibody or fragment of embodiment 25, wherein the antibody or fragment comprises:
(a) a bispecific antibody, optionally wherein the bispecific antibody is in a format selected from the group consisting of a bispecific IgG (BsIgG), an appended IgG, a bispecific antibody fragment, a bispecific fusion protein, and a bispecific antibody conjugate; or
(b) a trispecific antibody.

34. The antibody or fragment of embodiment 25, wherein the antibody or fragment comprises a bispecific T-cell engager (BiTE).

35. The antibody or fragment of embodiment 25, wherein the antibody or fragment comprises a chimeric antigen receptor (CAR).

36. An immunoconjugate comprising the antibody or fragment of any one of embodiments 25-32 and a detectable marker or cytotoxic agent.

37. The immunoconjugate of embodiment 36, wherein the immunoconjugate comprises a detectable marker, optionally wherein the detectable marker is selected from the group consisting of a radioisotope, a metal chelator, an enzyme, a fluorescent compound, a bioluminescent compound, and a chemiluminescent compound.

38. The immunoconjugate of embodiment 36, wherein the immunoconjugate comprises a cytotoxic agent, optionally wherein the cytotoxic agent is selected from the group consisting of an alkylating agent, an antimetabolite, an mitotic inhibitor, an antineoplastic antibiotic, a radionuclide, and a toxin.

39. An isolated nucleic acid encoding the antibody or fragment of any one of embodiments 25-35.

40. An expression cassette comprising the nucleic acid of embodiment 39, in operable combination with a regulatory sequence.

41. A host cell comprising the expression cassette of embodiment 40 or an expression vector comprising the expression cassette.

42. A method for the production of a recombinant antibody or fragment thereof, the method comprising:
a) culturing the host cell of embodiment 41 under conditions suitable for expression of the antibody or fragment; and
b) recovering the antibody or fragment from the host cell or its cell culture supernatant.

43. A method for characterizing a biopsy sample from a patient, comprising:
a) contacting the sample with the antibody or fragment of any one of embodiments 25-32 or the immunoconjugate of embodiment 37; and
b) detecting the binding of the antibody or fragment to cells of the sample, wherein the patient has received an effective amount of ARS-1620.

44. A method for treating cancer, the method comprising:
administering to a patient with cancer an effective amount of the antibody or fragment of any one of embodiments 25-35 or the immunoconjugate of embodiment 38, wherein the patient has received or is receiving an effective amount of ARS-1620.

45. The method of embodiment 44, wherein the cancer is an advanced solid tumor, optionally wherein the advanced solid tumor is a metastatic tumor or unresectable tumor.

46. The method of embodiment 44, wherein the cancer is a carcinoma.

47. The method of embodiment 46, wherein the carcinoma is a non-small cell lung carcinoma (NSCLC), a colorectal adenocarcinoma (CRC), or a pancreatic adenocarcinoma.

48. The method of any one of embodiments 44-47, wherein a kirsten rat sarcoma virus homolog (Kras) with a G12C mutation is expressed in cells of the cancer.

49. A composition for use in a method for treating cancer in a patient, comprising: an effective amount of the antibody or fragment of any one of embodiments 25-35 or the immunoconjugate of embodiment 38, wherein the patient has received or is receiving an effective amount of ARS-1620, and a kirsten rat sarcoma virus homolog (Kras) with a G12C mutation is expressed in cells of the cancer.

50. A radioisotope-labeled antibody or antibody fragment that specifically binds to VVVGAC (510) GVGK (SEQ ID NO:100), wherein 510 is AMG-510 (sotorasib), and the antibody or antibody fragment comprises:
(a) a light chain variable region comprising a CDRL1 of SEQ ID NO:49, a CDRL2 of SEQ ID NO:50, and a CDRL3 of SEQ ID NO:51, and a heavy chain variable region comprising a CDRH1 of SEQ ID NO:52, a CDRH2 of SEQ ID NO:53, and a CDRH3 of SEQ ID NO:54;
(b) a light chain variable region comprising a CDRL1 of SEQ ID NO:57, a CDRL2 of SEQ ID NO:58, and a CDRL3 of SEQ ID NO:59, and a heavy chain variable region comprising a CDRH1 of SEQ ID NO:60, a CDRH2 of SEQ ID NO:61, and a CDRH3 of SEQ ID NO:62;
(c) a light chain variable region comprising a CDRL1 of SEQ ID NO:65, a CDRL2 of SEQ ID NO:66, and a CDRL3 of SEQ ID NO:67, and a heavy chain variable region comprising a CDRH1 of SEQ ID NO:68, a CDRH2 of SEQ ID NO:69, and a CDRH3 of SEQ ID NO:70;
(d) a light chain variable region comprising a CDRL1 of SEQ ID NO:73, a CDRL2 of SEQ ID NO:74, and a CDRL3 of SEQ ID NO:75, and a heavy chain variable region comprising a CDRH1 of SEQ ID NO:76, a CDRH2 of SEQ ID NO:77, and a CDRH3 of SEQ ID NO:78;
(e) a light chain variable region comprising a CDRL1 of SEQ ID NO:81, a CDRL2 of SEQ ID NO:82, and a CDRL3 of SEQ ID NO:83, and a heavy chain variable region comprising a CDRH1 of SEQ ID NO:84, a CDRH2 of SEQ ID NO:85, and a CDRH3 of SEQ ID NO:86; or
(f) a light chain variable region comprising a CDRL1 of SEQ ID NO:89, a CDRL2 of SEQ ID NO:90, and a CDRL3 of SEQ ID NO:91, and a heavy chain variable region comprising a CDRH1 of SEQ ID NO:92, a CDRH2 of SEQ ID NO:93, and a CDRH3 of SEQ ID NO:94.

51. The antibody or antibody fragment of embodiment 50, wherein the antibody or fragment comprises: a light chain variable region comprising a CDRL1 of SEQ ID NO:49, a CDRL2 of SEQ ID NO:50, and a CDRL3 of SEQ ID NO:51, and a heavy chain variable region comprising a CDRH1 of SEQ ID NO:52, a CDRH2 of SEQ ID NO:53, and a CDRH3 of SEQ ID NO: 54.

52. The antibody or antibody fragment of embodiment 51, wherein the antibody or fragment comprises: a light chain variable region comprising the amino acid sequence of SEQ ID NO: 47 and a heavy chain variable region comprising the amino acid sequence of SEQ ID NO:48.

53. The antibody or antibody fragment of embodiment 52, wherein the antibody or fragment comprises: a light chain comprising the amino acid sequence of SEQ ID NO:11 and a heavy chain comprising the amino acid sequence of SEQ ID NO:17, or a light chain comprising an amino acid sequence having at least 95% sequence identity to SEQ ID NO:11 and a heavy chain comprising an amino acid sequence having at least 95% sequence identity to SEQ ID NO: 17.

54. The antibody or antibody fragment of any one of embodiments 50-53, wherein the antibody or antibody fragment is a recombinant IgG₁ antibody.

55. The antibody or antibody fragment of any one of embodiments 50-54, wherein the radioisotope is a therapeutic radioisotope selected from the group consisting of ⁶⁷Cu, ⁹⁰Y, ¹³¹I, ¹⁵³Sm, ¹⁷⁷Lu, ²¹¹At, ²¹²Pb, ²²³Ra, ²²⁵Ac, and ²²⁷Th.

56. The antibody or antibody fragment of embodiment 55, wherein the therapeutic radioisotope is ¹⁷⁷Lu or ²²⁵Ac.

57. The antibody or antibody fragment of any one of embodiments 50-54, wherein the radioisotope is a diagnostic radioisotope selected from the group consisting of ¹⁸F, ⁴⁴Sc ⁶⁴Cu, ⁶⁸Ga, ⁸⁹Zr, and ¹²⁴I.

58. The antibody or antibody fragment of embodiment 57, wherein the diagnostic radioisotope is ⁸⁹Zr.

59. A method for treating a cancer expressing a kirsten rat sarcoma virus homolog (Kras) with a G12C mutation (Kras G12C cancer), the method comprising:
administering to a patient with a Kras G12C cancer an effective amount of the antibody or antibody fragment of embodiment 55 to treat the cancer,
wherein the patient has received or is receiving an effective amount of sotorasib.

60. The method of embodiment 59, wherein the patient is HLA-A*03.01-positive.

61. The method of embodiment 60, further comprising prior to the administering step, HLA-typing the patient.

62. The method of any one of embodiments 59-61, wherein the cancer is an advanced solid tumor, optionally wherein the advanced solid tumor is a metastatic tumor or an unresectable tumor.

63. The method of any one of embodiments 59-62, wherein the cancer is a carcinoma.

64. The method of embodiment 63, wherein the carcinoma is a non-small cell lung carcinoma (NSCLC), a colorectal adenocarcinoma (CRC), or a pancreatic adenocarcinoma.

65. The method of any one of embodiments 59-64, further comprising prior to the administering step, determining the antibody or antibody fragment binds to cells of the Kras G12C cancer by immunohistochemistry analysis of a biopsy obtained from the subject.

66. A composition for use in a method for treating cancer in a patient, comprising: an effective amount of the antibody or fragment of claim 55, wherein the patient has received or is receiving an effective amount of sotorasib (AMG-510), and a kirsten rat sarcoma virus homolog (Kras) with a G12C mutation is expressed in cells of the cancer.

67. The use of embodiment 66, wherein the patient is HLA-A*03.01-positive.

68. The use of embodiment 66 or 67, wherein the cancer is an advanced solid tumor.

69. The use of any one of embodiments 66-68, wherein the cancer is a carcinoma.

70. The use of embodiment 69, wherein the carcinoma is a non-small cell lung carcinoma (NSCLC), a colorectal adenocarcinoma (CRC), or a pancreatic adenocarcinoma.

### EXAMPLES

The present disclosure is described in further detail in the following examples, which are not in any way intended to limit the scope of the disclosure unless otherwise indicated in the claims. The attached figures are meant to be considered as integral parts of the specification and description of the disclosure. The following examples are offered to illustrate, but not to limit the claimed disclosure.

In the experimental disclosure which follows, the following abbreviations apply: AMG510 or AMG-510 (6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one, also known as sotorasib); B2m (beta-2 microglobulin); BiTE (Bispecific I cell Engager); BLI (biolayer interferometry); BsAb (bispecific antibody); CAR (chimeric antigen receptor); CDR (complementarity determining region); CheCmATE (Chemically Controlled monoclonal Antibody Target Engagement); DSF (differential scanning fluorimetry); ELISA (enzyme-linked immunosorbent assay); Fab (antigen-binding fragment); IgG (immunoglobulin G); MHC (major histocompatibility complex); MHC I (MHC class I); pMHC (peptide MHC); and TCR (T cell receptor).

### Example 1. Covalent Inhibitors of K-Ras (G12C) Induce MHC-I Presentation of Haptenated Peptide Neoepitopes Targetable by Immunotherapy

This example describes the isolation and characterization of antigen-binding fragments (Fabs) that react with a tumor-specific neoantigen. The neoantigen is produced by attachment of an oncoprotein-specific hapten to an oncoprotein (KRas G12C), followed by processing and presentation on the cell surface as a peptide-MHC (pMHC). In this example, the hapten is a small molecule drug, namely a covalent inhibitor of KRas G12C (e.g., ARS1620 or AMG510).

### Materials and Methods

Identification of Fabs from Phage Display Libraries. Neoantigen-specific Fabs were isolated using the Craik Jab panning platform with a human, naïve Fab-phage display library with a diversity of 4×10¹⁰ (Duriseti et al., J Biol Chem 285, 26878-26888, 2010). The Fabs were panned against a minimal antigen of a KRas G12C 10-mer peptide-biotin conjugate labeled with ARS1620 or AMG510 on cysteine, or a complex of the labeled KRas peptide presented by HLA-A*03:01, by immobilization using streptavidin magnetic beads. Negative selection was done in rounds 3 and 4 with the cognate K-Ras peptide without ARS1620 or AMG510 modification in the case of peptide-based panning or with a complex of the unlabeled KRas peptide presented by HLA-A*03:01. After four rounds of selection, individual clones were screened in an ELISA for binding to the target antigen (labeled KRas peptide alone or as MHC-I complex). Clones with a positive signal were sequenced and unique clones were expressed in BL21(DE3) E. coli and purified for further analysis.

Kinetic measurements via Octet. Kinetic constants for Fabs were determined using an Octet RED384 instrument (ForteBio). For screens, a single concentration of Fab (100 nM) was tested, and for kinetic characterization Fabs were tested at several concentrations, as noted in the relevant figure. Biotinylated peptides (1 μM in 1% BSA/PBS) or biotinylated MHC-I complexes (200 nM in 1% BSA/PBS) were immobilized on ForteBio streptavidin SA biosensors for all assays. All measurements were performed in 1% BSA PBS pH 7.4 in 384 well plates. Data were analyzed using a 1:1 interaction model with global fitting on the ForteBio data analysis software (9.0.0.6). KD values were determined by the fitting of either equilibrium or maximum response (nm) as a function of Fab concentration.

Differential Scanning Fluorimetry. All DSF measurements were made on a Bio-Rad C1000 qPCR system in FRET mode. Fab (2 μM) was added to either DMSO or antigen (50 μM) with 5× SYPRO dye and plated in triplicate in a white, 96 well PCR plate in PBS. MHC-I complexes (2 μM) were combined with 5× SYPRO dye in Tris buffer (20 mM, pH=7.0, 150 mM NaCl) and plated in quadruplicate in a white, 96 well PCR plate. The temperature was initially kept at 23° C. for five minutes before slow ramping in 0.5° C. increments every 30 s. Raw data was normalized from 0 to 1 before fitting as described above.

MHC-I refolding and purification. MHC heavy chain and beta-2 microglobulin (B2m) were expressed and purified following the protocol of Rodenko et al. (Nat. Protoc., 1, 1120-1132, 2006). Refolding reactions were performed with various peptides of interest in refolding buffer (100 mM Tris pH 8.0, 400 mM L-Arginine.HCl, 5 mM reduced glutathione, 0.5 mM oxidized glutathione, 2 mM EDTA, and complete protease inhibitor cocktail (Roche)). Briefly, B2m (2 μM) and peptide (10 μM) were diluted into refolding buffer, then denatured heavy chain was added to 1 μM. Reactions proceeded at 10° C. overnight for ELISA assays. MHC-I ELISAs. Black, 384 well Nunc Maxisorp plates were coated with 50 μL of the anti-heavy chain antibody W6/32 (Bio X Cell Cat #BE0079, RRID:AB_1107730) at 5 μg/mL in PBS overnight. The plate was washed twice with PBS (100 μL) and blocked with 3% BSA PBS (120 μL) for 1 hour at room temperature. Plates were then washed three times with 0.05% Tween-20 PBS (PBST) (100 μL). Refolded MHC complexes (crude or FPLC purified) were diluted 10× into 1% BSA PBS and 50 μL added to wells in quadruplicate. Plates were incubated at room temperature with shaking for 1 hour, then washed three times with 1% BSA PBS (100 μL). Free ARS1620 was combined with either P1A4 IgG-HRP conjugate or P2B2 IgG-HRP conjugate and exposed to immobilized, refolded complexes for 1.5 hours at room temperature for the competition ELISA. Free ARS1620 concentration ranged from 0.4 nM to 1 μM for P1A4 and 24 nM to 25 μM for P2B2. Complexes were detected with either the anti-B2m HRP conjugate (Santa Cruz Biotechnology Cat #sc-13565, RRID:AB 626748) to measure total MHC-I complexes, or with a P1A4 IgG-HRP conjugate or P2B2 IgG-HRP conjugate to detect targetable ARS1620 in these complexes. For both antibodies, 50 μL of 1 μg/mL antibody solution in 1% BSA PBS was added to each well. Plates were incubated at room temperature with shaking for 1 hour. Plates were then washed three times with PBST and three times with PBS. 50 μL of the HRP substrate QuantaBlu (Thermo Fisher Scientific) was then added and activity measured continuously for 45 minutes at 325/420 nm in a BioTek Synergy H4 plate reader. Endpoint fluorescence readings were also taken after I hour of development.

The amino acid sequences of the synthetic peptides used to identify and/or characterized the Fabs of interest are shown in Table 1-1.

TABLE 1-1
Peptides^{∧}

| Peptide | Origin | Sequence |
|---|---|---|
| G12C | Kras | GACGVGKSAL (SEQ ID NO: 1) |
| G12C-1620 (S-atropisomer) | Kras | GAC(1620)GVGKSAL (SEQ ID NO: 2) |
| G12C-1620e* (R-atropisomer) | Kras | GAC(1620e*)GVGKSAL (SEQ ID NO: 3) |
| PolyG-1620 (S-atropisomer) | control | GGC(1620)GGGGGG-Nle (SEQ ID NO: 4) |
| CEF1 | influenza | GILGFVFTL (SEQ ID NO: 101) |
| ALN (MHC 15) | yellow fever | ALNTITNLK (SEQ ID NO: 95) |
| K5 | Kras | KLVVVGACGV (SEQ ID NO: 96) |
| K5-1620 | Kras | KLVVVGAC(1620)GV (SEQ ID NO: 97) |
| V7 | Kras | VVVGACGVGK (SEQ ID NO: 98) |
| V7-1620 | Kras | VVVGAC(1620)GVGK (SEQ ID NO: 99) |
| V7-510 | Kras | VVVGAC(510)GVGK (SEQ ID NO: 100) |

^{∧}C(1620) = ARS1620 modification of cysteine (S-atropisomer unless otherwise indicated); and C(510) = AM510 modification of cysteine.

### Results

ARS1620-modified KRas G12C peptides are competent for antigen presentation. KRas G12C is one of the most prevalent oncogenic driver mutations in lung and colon cancer. While covalent inhibitors (e.g. Sotorasib/AMG510, Adagrasib, JNJ-74699157, LY3499446, ARS1620) that specifically react with the acquired cysteine (Cys12) residue have been reported to rapidly engage cellular KRas (G12C) proteins and drive tumor regression in mouse models and human cancer patients, not all patients with a KRas G12C mutation respond to KRas (G12C) inhibitors. Clinical resistance to both Sotorasib and Adagrasib have already been observed, with various mechanisms including mutations on the WT KRas allele in trans. However, most resistant tumors retain the expression of KRas G12C. Therefore, an immunotherapy that targets the KRas G12C mutation is desirable to circumvent these resistance mechanisms and benefit a large patient population.

Although MHC-I presentation of mutant KRas peptides has been observed in patients, it remained unknown whether a covalently attached inhibitor would interfere with antigen processing and subsequent binding to MHC-I complexes. This question was first addressed using the investigational KRas (G12C) inhibitor ARS1620, as it was an advanced drug candidate (FIG. 1A). Two common MHC-I alleles, HLA-A*02:01 and HLA-A*03:01, for which K-Ras peptide epitopes containing the mutant cysteine have been reported in the Immune Epitope DataBase (IEDB) were the focus of the studies described herein. Peptides K5-1620 (SEQ ID NO: 97) and V7-1620 (SEQ ID NO:99) comprising an ARS1620-modified cysteine, were synthesized by solid-phase peptide synthesis followed by base-mediated Michael addition to introduce ARS1620 onto Cys12. These two ARS1620-modified peptides readily formed functional MHC-I complexes with HLA-A*02:01 and HLA-A*03:01, respectively, in an in vitro MHC refolding assay in which complex formation was detected by a sandwich ELISA (FIG. 1B). Furthermore, the K5-1620 peptide stabilized HLA-A*02:01 expression on the surface of T2 cells, which express HLA-A*02:01 but are deficient in the transporter associated with antigen processing (TAP) genes and only form functional MHC complexes with exogenously supplied cognate peptides (FIG. 1C). The thermostability of HLA-A*02:01 complexes loaded with various peptides was assessed by measuring denaturation of folded pMHC using differential scanning fluorimetry (DSF) (FIG. 1D). Together, these results confirm that K-Ras peptides can be bound by two common MHC Class I alleles and that inhibitor modification of the peptide is tolerated by the peptide-binding cleft in the alleles examined.

Identification and characterization of ARS1620-modified KRas G12C peptide-specific Fabs. Five unique Fabs were initially isolated and characterized (see, WO 2021/014417 of Craik et al.). All five clones showed specific and high-affinity binding to an ARS1620-labeled peptide with affinities ranging from 14 nM to 51 nM. One clone, P1A4, featured a relatively short heavy chain complementarity-determining region 3 (CDR3) as well as a remarkable selectivity for the S atropisomer of ARS1620. P1A4 was chosen as a comparator for new Fabs described herein.

Two further clones, P2B2 and P1C10, were identified as having high affinity for V7-ARS1620 when presented in the HLA-A*03:01 MHC-I complex (43 nM and 59 nM, respectively). The amino acid sequences of regions of the anti-ARS1620 Fabs, P1A4, P2B2 and P1C10 are shown in Table 1-2.

TABLE 1-2
Anti-ARS-1620 Fab Sequences

| Region | P1A4 Sequence |
|---|---|
| LCVR | QSVVTQPPSVSAAPGQEVTISCSGSSSNIGNNYVSWYQHLPGTAPKLLIYDSDKRPSGIPDRFSG |
| | SKSGTSATLGVTGLQTGDEADYYCGTWDSSLSAVMFGGGTKLTVL |
| | (SEQ ID NO: 23) |
| HCVR | QVQLQESGGGLVQPGRSLRLSCAASGFTFSSYAMHWVRQAPGKGLEWVAVISYDGSNKYYADSVK |
| | GRFTISRDNSKNTLYLQMNSLRAEDTAVYYCARDYDWQYFDYWGQGTLVTVSS |
| | (SEQ ID NO: 24) |
| CDRL1 | SGSSSNIGNNYVS (SEQ ID NO: 25) |
| CDRL2 | DSDKRPS (SEQ ID NO: 26) |
| CDRL3 | GTWDSSLSAVM (SEQ ID NO: 27) |
| CDRH1 | GFTFSSYAMH (SEQ ID NO: 28) |
| CDRH2 | VISYDGSNKY (SEQ ID NO: 29) |
| CDRH3 | DYDWQYFD (SEQ ID NO: 30) |
| Region | P282 Sequence |
| LCVR | QSVVTQPPSVSAAPGHKVSISCSGSNSNIGHNYVSWYQQFPGTAPKLLVYRDDNRPSEVPDRFSG |
| | SKSGTSATLGITGLQTADEADYYCGTWDSTLQHVVFGGGTKLTVL |
| | (SEQ ID NO: 31) |
| HCVR | QVQLVESGGGVVQPGRYLRLSCATSGFTFSNYAMHWVRQAPGKGLEWVALISYDGSNKYYADSVK |
| | GRFTISRDKSTNALYLQMNSLRVEDTAVYYCARERVPWDYYGMDVWGQGTTVTVSS |
| | (SEQ ID NO: 32) |
| CDRL1 | SGSNSNIGHNYVS (SEQ ID NO: 33) |
| CDRL2 | RDDNRPS (SEQ ID NO: 34) |
| CDRL3 | GTWDSTLQHVV (SEQ ID NO: 35) |
| CDRH1 | GFTFSNYAMH (SEQ ID NO: 36) |
| CDRH2 | LISYDGSNKYY (SEQ ID NO: 37) |
| CDRH3 | ERVPWDYYGMDV (SEQ ID NO: 38) |
| Region | P1C10 Sequence |
| LCVR | QSVVTQPPSVSAAPGQKVTISCSGSSSNIGNHYVSWYQQLPGTAPKLLIYDDGKRPSGIPDRFSG |
| | SKSGTSATLGITGLQTGDEADYYCATWDNSLSVVLFGGGTKLTVL |
| | (SEQ ID NO: 39) |
| HCVR | QVQLVQSGGGLVKPGGSLRLSCAASGFTFSSYSMNWVRQAPGKGLEWVSYISSRGRTIYYADSVK |
| | GRFTISRDNAKNSLYLQMNSLRAEDTAVYYCVRDVPGVWGFAYWGQGTLVTVSS |
| | (SEQ ID NO: 40) |
| CDRL1 | SGSSSNIGNHYVS (SEQ ID NO: 41) |
| CDRL2 | DDGKRPS (SEQ ID NO: 42) |
| CDRL3 | ATWDNSLSVVL (SEQ ID NO: 43) |
| CDRH1 | GFTFSSYSMN (SEQ ID NO: 44) |
| CDRH2 | YISSRGRTIYY (SEQ ID NO: 45) |
| CDRH3 | DVPGVWGFAY (SEQ ID NO: 46) |

P2B2 and P1C10 were specific to the modified V7 peptide (SEQ ID NO:99), with lower affinity for the ARS1620-modified K5 peptide (SEQ ID NO:97) when presented in the A*02:01 MHC-I complex (470 nM and 320 nM) (FIG. 2A, 2C). P2B2 displayed lower affinity for the free V7-ARS peptide (180 nM), while P1C10 showed similar affinity for the free V7-ARS peptide (56 nM) as when presented (59 nM) (FIG. 2A, 2C). Thermal shift assays indicated that both clones bound tighter to ARS1620 and reduced ARS1620 than to the R atropisomer of ARS1620 or AMG-510 (Sotorasib) (FIG. 2B, 2C).

As shown by competition ELISA with ARS1620, the specificity of Fab P2B2 for the modified peptide when bound to the MHC-I complex (FIG. 4B) is a demonstrable improvement over P1A4, which binds free ARS1620 with high affinity (FIG. 4A).

Identification and characterization of AMG510-modified KRas G12C peptide-specific Fabs. Six distinct clones were identified with high affinities for KRas (G12C) epitopes modified by AMG510. The amino acid sequences of regions of the anti-AMG510 Fabs, P1B7, P1H4, P2B6, P2E3, P1E5 and P2C1 are shown in Table 1-3.

TABLE 1-3
Anti-AMGS10 (Sotorasib) Fab Sequences

| Region | P1B7 Sequence |
|---|---|
| LCVR | LFAIPLVVPFYSHSAQSALTQPPSVSGSPGQSVTISCTGSSSDVGHYNRVSWYQQSPGTAPKLMI |
| | YDVSNRPSGAFSRFSGSRSGNTASLTISGLQPEDEADYYCSSHRTGYTVEVFGTGTKVTVLGQPK |
| | ANPTVTLFPPSSEELQANKATLVCLISDFYPGAVTVAWKADGSPVKAGVETTKPSKQSNNKYAAS |
| | SYLSLTPEQWKSHRSYSCQVTHEGSTVEKTVAPAECS |
| | (SEQ ID NO: 47) |
| HCVR | QVQLQQSGPGLVKPSQTLSLTCAISGDSVSSNSVAWNWIRQSPSRGLEWLGRTYYRSKWYSDYAV |
| | SVKSRITINPDTSKNQFSLQLTSMTPEDTAVYYCAREGVTMGPGAFDIWGQGTMVTVSSASTKGP |
| | SVFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCAAA |
| | (SEQ ID NO: 48) |
| CDRL1 | TGSSSDVGHYNRVS (SEQ ID NO: 49) |
| CDRL2 | DVSNRPS (SEQ ID NO: 50) |
| CDRL3 | SSHRTGYTVEV (SEQ ID NO: 51) |
| CDRH1 | GDSVSSNSVAWN (SEQ ID NO: 52) |
| CDRH2 | RTYYRSKWYSD (SEQ ID NO: 53) |
| CDRH3 | EGVTMGPGAFD (SEQ ID NO: 54) |
| Region | P1H4 Sequence |
| LCVR | LFAIPLVVPFYSHSALETTLTQSPGTLSLSPGERATLSCRASQSVSSSYLAWYQQKPGQAPRLLI |
| | YGASSRATGIPDRFSGSGSGTDFTLTISRLEPGDFAVYYCQQYGTSPITFGQGTRLEIKRTVAAP |
| | SVFIFPPSDEQLKSGTASVVCLLNNFYPREAKVQWKVDNALQSGNSQESVTEQDSKDSTYSLSST |
| | LTLSKADYEKHKVYACEVTHQGLSSPVTKSFNRGEC |
| | (SEQ ID NO: 55) |
| HCVR | EVQLVQSGAEVKKPGSSVKVSCKASGGTFSSYAISWVRQAPGQGLEWMGGIIPIFGTANYAQKFQ |
| | GRVTITADKSTSTAYMELSSLRSEDTAVYYCAFNYYDSSGYSDYWGQGTLVTVSSASTKGPSVFP |
| | LAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCAAA |
| | (SEQ ID NO: 56) |
| CDRL1 | RASQSVSSSYLA (SEQ ID NO: 57) |
| CDRL2 | GASSRAT (SEQ ID NO: 58) |
| CDRL3 | QQYGTSPIT (SEQ ID NO: 59) |
| CDRH1 | GGTFSSYAIS (SEQ ID NO: 60) |
| CDRH2 | GIIPIFGTAN (SEQ ID NO: 61) |
| CDRH3 | NYYDSSGYSD (SEQ ID NO: 62) |
| Region | P2B6 Sequence |
| LCVR | LFAIPLVVPFYSHSALDVVMTQSPLSLPVTPGEPASISCRSSQSLLRNNGYNYLDWYLQKPGQSP |
| | QLLIYLGSNRASGVPDRFSGSGSGRDFTLKISRVEAEDVGIYYCMQGAHWPYTFGQGTNLEISRT |
| | VAAPSVFIFPPSDEQLKSGTASVVCLLNNFYPREAKVQWKVDNALQSGNSQESVTEQDSKDSTYS |
| | LSSTLTLSKADYEKHKVYACEVTHQGLSSPVTKSFNRGEC |
| | (SEQ ID NO: 63) |
| HCVR | QLQLQESGPGLVKPSETLSLTCTVSGVSISSRSDHWGWVRQPPGKGLEWIGSISYSGSTYYNPSL |
| | KSRVTISVDTSKNQLSLKLTSVTAADTAVYYCARRALAQQLVMDVWGQGTTVTVSSASTKGPSVF |
| | PLAPSSKSTSGGTAALGCLVKDYFPEPVTVSWNSGALTSGVHTFPAVLQSSGLYSLSSVVTVPSS |
| | SLGTQTYICNVNHKPSNTKVDKKVEPKSCAAA |
| | (SEQ ID NO: 64) |
| CDRL1 | RSSQSLLRNNGYNYLD (SEQ ID NO: 65) |
| CDRL2 | LGSNRAS (SEQ ID NO: 66) |
| CDRL3 | MQGAHWPYT (SEQ ID NO: 67) |
| CDRH1 | GVSISSRSDHWG (SEQ ID NO: 68) |
| CDRH2 | SISYSGSTY (SEQ ID NO: 69) |
| CDRH3 | RALAQQLVMD (SEQ ID NO: 70) |
| Region | P2E3 Sequence |
| LCVR | LFAIPLVVPFYSHSALDVVMTQSPLSLPVTPGEAASISCRSSQSLLHSNGYTYLDWYLQKPGQSP |
| | RLLIYSGSNRASGVPDRFSGSGSGTDFTLKISRVEAEDVGVYYCMQGTHWPYTFGQGTRLEIKRT |
| | VAAPSVFIFPPSDEQLKSGTASVVCLLNNFYPREAKVQWKVDNALQSGNSQESVTEQDSKDSTYS |
| | LSSTLTLSKADYEKHKLYACEVTHQGLSSPVTKSFNRGEC |
| | (SEQ ID NO: 71) |
| HCVR | QVQLQESGPGLVKPSETLSLTCTVSGGSVSSSSYYWGWIRQPPGEGLEWIGSIYYSGSTYYNPSL |
| | KSRVTISVDTSKNQFSLKLSSVTAADTAVYYCATRLRGSFDYWGQGTLVTVSSASTKGPSVFPLA |
| | PSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCAAA |
| | (SEQ ID NO: 72) |
| CDRL1 | RSSQSLLHSNGYTYLD (SEQ ID NO: 73) |
| CDRL2 | SGSNRAS (SEQ ID NO: 74) |
| CDRL3 | MQGTHWPYT (SEQ ID NO: 75) |
| CDRH1 | GGSVSSSSYYWG (SEQ ID NO: 76) |
| CDRH2 | SIYYSGSTY (SEQ ID NO: 77) |
| CDRH3 | RLRGSFD (SEQ ID NO: 78) |
| Region | P1E5 Sequence |
| LCVR | LFAIPLVVPFYSHSALDVVMTQSPLSLPVTPGEPASISCRSSQSLLHSNGYNYLDWYLQKPGQSP |
| | QLLIYLGSNRASGVPDRFSGSGSGTDFTLKISRVEAEDVGVYYCMQGTHWPPGYTFGQGTKVEIN |
| | RTVAAPSVFIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 79) |
| HCVR | QVQLQESGGGVVQPGRSLRLSCAASGFTFSSYEMNWVRQAPGKGLEWVAVISYDGSNKYYADSVK |
| | GRFTISRDNSKNTLYLQMNSLRAEDTAVYYCARDAYSGWFDYWGQGTLVTVSSASTKGPSVFPLA |
| | PSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCAAA |
| | (SEQ ID NO: 80) |
| CDRL1 | RSSQSLLHSNGYNYLD (SEQ ID NO: 81) |
| CDRL2 | LGSNRAS (SEQ ID NO: 82) |
| CDRL3 | MQGTHWPPGYT (SEQ ID NO: 83) |
| CDRH1 | GFTFSSYEMN (SEQ ID NO: 84) |
| CDRH2 | VISYDGSNKY (SEQ ID NO: 85) |
| CDRH3 | DAYSGWFD (SEQ ID NO: 86) |
| Region | P2C1 Sequence |
| LCVR | LFAIPLVVPFYSHSAQSALTQPASVSGSPGQSITISCTGTSSDVGSYNLVSWYQQHPGKAPKLMI |
| | YDVSNRPSGVSNRFSGSKSGNTASLTISGLQAEDEADYYCSSYTSSSTLVFGTGTNVTVLGQPKA |
| | NPTVTLFPPSSEELQANKATLVCLISDFYPGAVTVAWKADGSPVKAGVETTKPSKQSNNKYAASS |
| | YLSLTPEQWKSHRSYSCQVTHEGSTVEKTVAPTECS |
| | (SEQ ID NO: 87) |
| HCVR | EVQLVQSGAEVKKPGSSVKVSCKASGGTFSSYAISWVRQAPGQGLEWMGIINPSGGSTSYAQKFQ |
| | GRVTMTRDTSTSTVYMELSSLRSEDTAVYYCARDLGTIFEANGAFDIWGQGTMVTVSSASTKGPS |
| | VFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCAAA |
| | (SEQ ID NO: 88) |
| CDRL1 | TGTSSDVGSYNLVS (SEQ ID NO: 89) |
| CDRL2 | DVSNRPS (SEQ ID NO: 90) |
| CDRL3 | SSYTSSSTLV (SEQ ID NO: 91) |
| CDRH1 | GGTFSSYAIS (SEQ ID NO: 92) |
| CDRH2 | IINPSGGSTS (SEQ ID NO: 93) |
| CDRH3 | DLGTIFEANGAFDI (SEQ ID NO: 94) |

The binding affinities of the six Fabs for V7-AMG510 when presented in the HLA-A*03:01 MHC-I complex range from 13 nM for P1E5 to 230 for P2C1 (FIG. 3A, FIG. 3B). None of the six clones showed significant binding to the V7 WT HLA-A*03:01 MHC-I complex up to luM. Some clones, such as P1E5, showed tight binding to the free modified V7-AMG510 peptide (54 nM), while others, like P1B7, displayed low affinity for the free peptide (4900 nM). Competition assays for binding to free AMG510 versus V7-AMG510 HLA-A*03:01 MHC-I complexes revealed that the five clones have IC₅₀ values ranging from 30 nM for P2E3 to more than 50,000 nM for P1B7 (FIG. 3B, FIG. 3C), where IC₅₀ values were determined by least-squares regression in GraphPad Prism 8.0. These antibodies widen the scope of drug discovery, and overcome the limitation of antibodies such as P1A4 that also bind free peptide with high affinity.

### Example 2. Characterization of Antibody Binding to Sotorasib-Labeled MHC Claims I Complexes

P1B7 is an antibody isolated from a naïve-human Fab-phage display library via a biopanning campaign against a Sotorasib-labeled, KRas G12C-derived major histocompatibility I (MHC 1) complex (V7-Sotorasib A*03:01). P1B7 shows exquisite specificity for the inhibitor-modified MHC I complex (Fab K_{D}=15 nM) over the cognate WT MHC I complex (binding not detected, Fab K_{D}>1 μM). It also shows greatly reduced affinity to the V7-Sotorasib peptide in the absence of the MHC I context (K_{D}=˜5 μM), confirming both a Sotorasib and MHC I dependence for its binding. First-generation antibodies, which bound the structurally related KRas G12C inhibitor ARS1620, suffered a liability in which free inhibitor competed for binding to the antibody, precluding their further characterization in vivo where circulating ARS1620 concentrations were high enough to completely saturate the antibody binding sites. P1B7 overcame this limitation and is highly resistant to competition with free Sotorasib (Fab IC₅₀>50 μM). This binding specificity profile confirms that binding of P1B7 is dependent on all members of the composite MHC I antigen, making it an ideal candidate for further study and therapeutic development.

### Example 3. Inhibition of Tumor Growth by Targeting Sotorasib-Labeled MHC I Complexes with Radiolabeled Antibodies

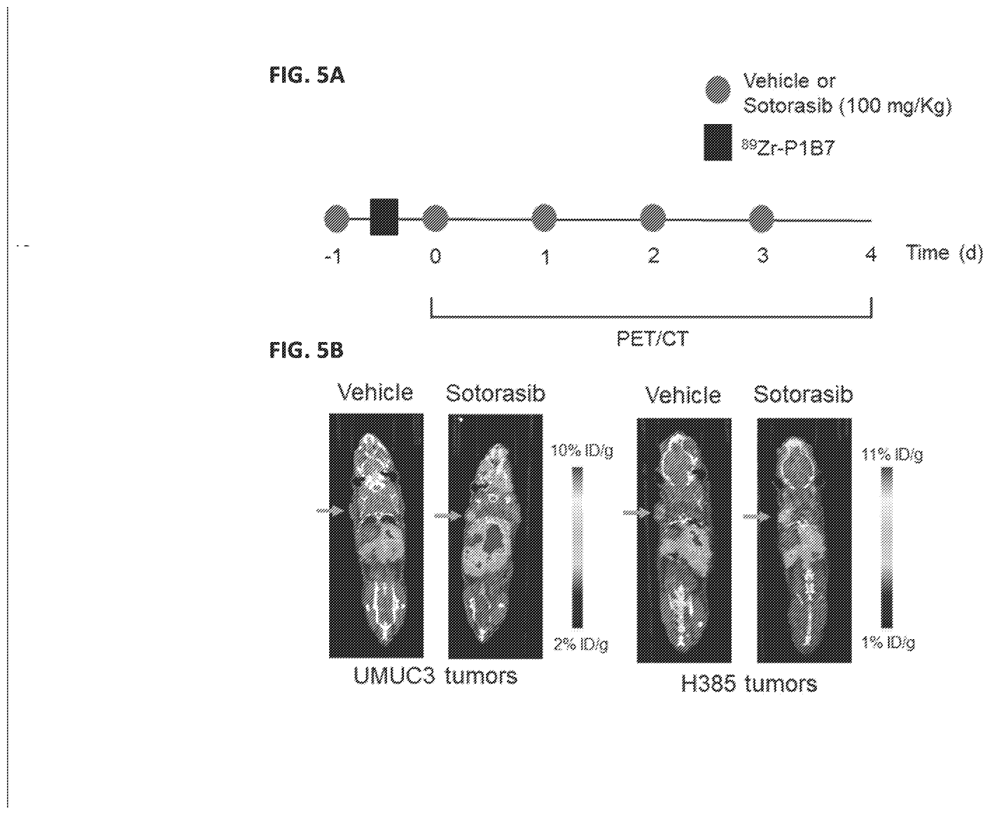

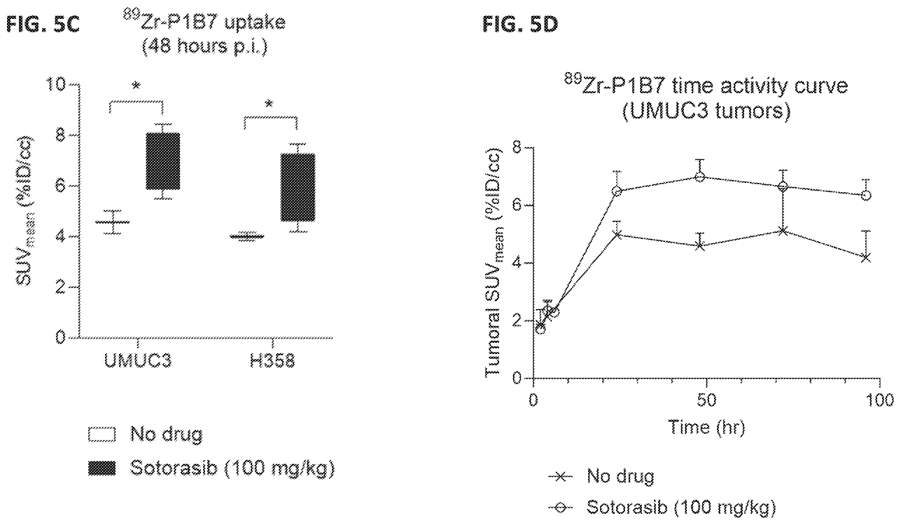

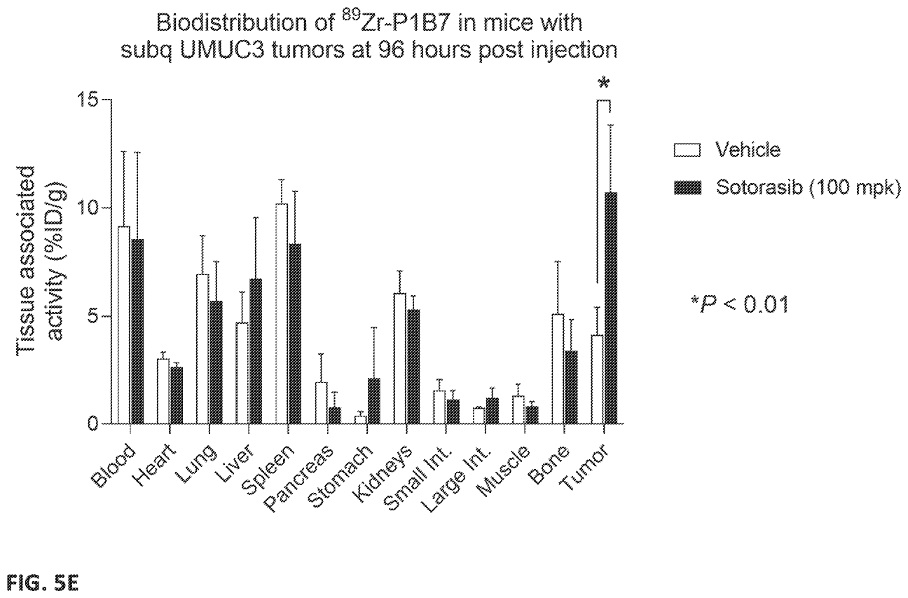

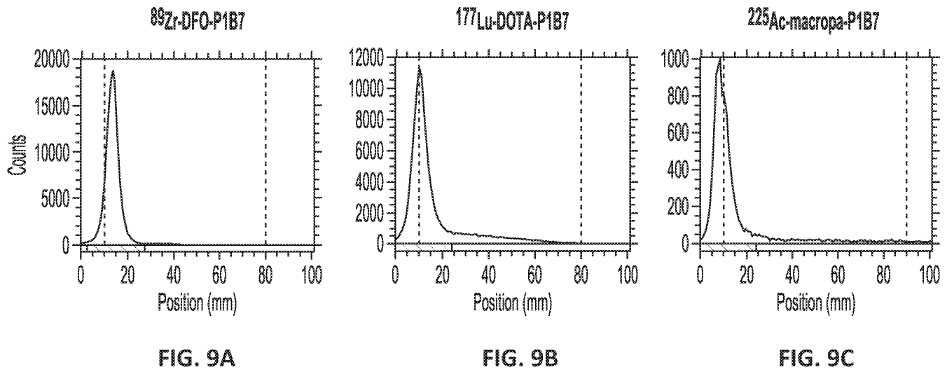

After established P1B7 as an antibody with favorable binding characteristics, its efficacy was tested in vivo using xenograft models of KRas G12C mutant cancer. To first confirm that P1B7 localizes to the tumor microenvironment upon Sotorasib treatment, a P1B7 IgG was converted to a radiopharmaceutical for imaging with positron emission tomography (PET) via lysine-based p-Bn-SCN-DFO coupling and subsequent loading with Zr-89 (FIG. 9A). Two human KRas G12C cell lines, UMUC3 (bladder carcinoma) and H358 (lung carcinoma), were implanted on the left flank and grown until tumors reached ˜200 mm³ volume. The two arms of this study were ⁸⁹Zr—P1B7 IgG alone and ⁸⁹Zr—P1B7 IgG with Sotorasib (100 mg/kg) treatment (n=4/arm). Mice were treated with Sotorasib (100 mg/kg) or vehicle (Saline) daily beginning at Day −1 by oral gavage and the ⁸⁹Zr—P1B7 IgG PET imaging agent via tail vein injection on Day 0 (FIG. 5A). After 48 hours, accumulation of the ⁸⁹Zr—P1B7 IgG PET imaging agent was apparent in both the UMUC3 and H358 tumors treated with Sotorasib, but not those treated with vehicle alone (FIG. 5B). Quantification of this localization confirms that ⁸⁹Zr—P1B7 IgG is significantly enriched (P<0.01) upon Sotorasib treatment only in xenografted tumors and not in any other tissue, thus confirming both Sotorasib and KRas G12C-dependent localization (FIG. 5C-5E).

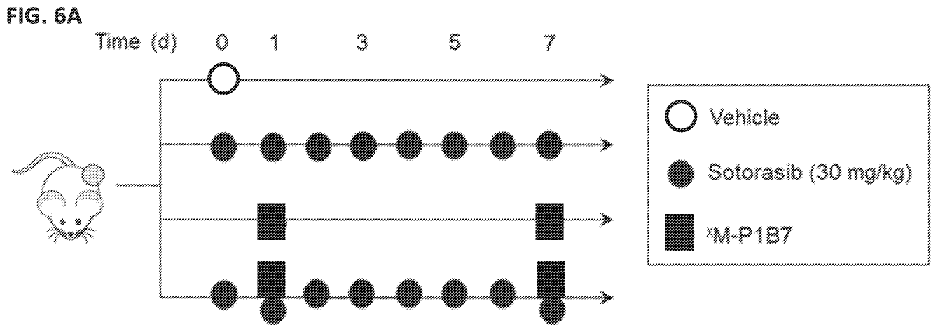

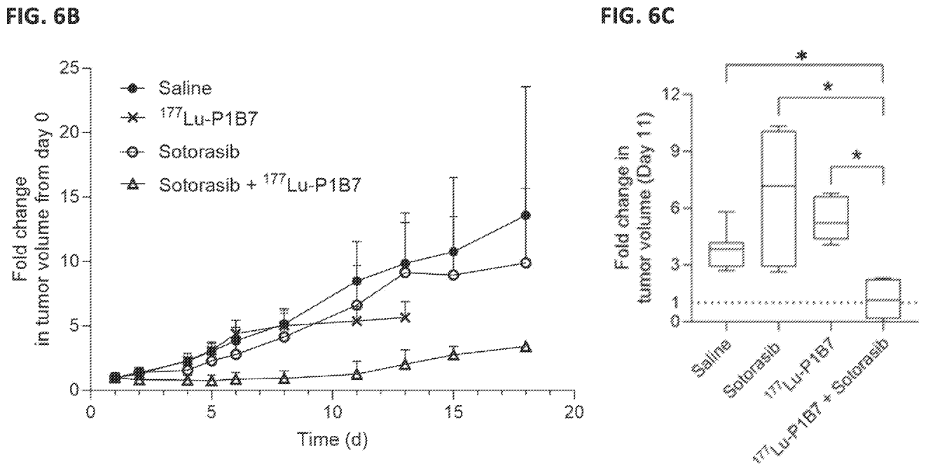

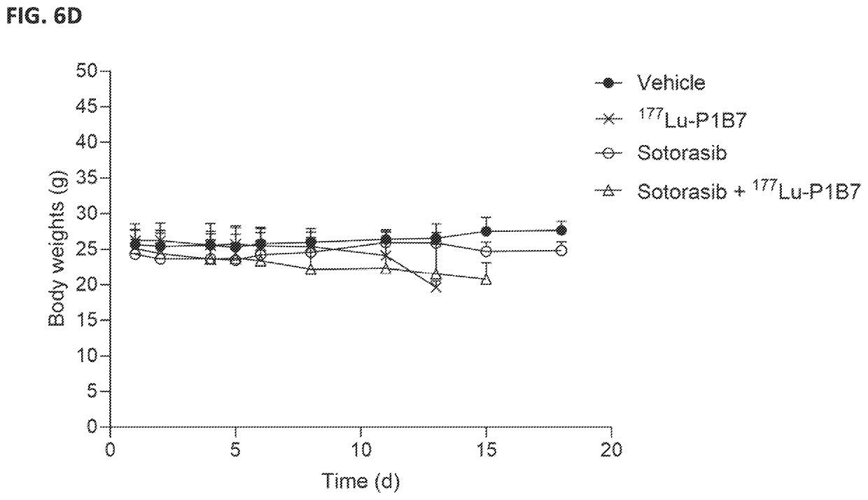

Having confirmed that a P1B7 IgG can localize to the tumor microenvironment upon Sotorasib treatment, whether P1B7 could have a therapeutic benefit as the basis of a radioligand therapy was tested. The P1B7 IgG was converted to a radioligand therapeutic through lysine-based p-Bn-SCN-DOTA coupling and subsequent loading with the β-emitter Lu-177 (FIG. 9B). Mice were split into four treatment arms (n=7 per arm): saline, Sotorasib (30 mg/kg), ¹⁷⁷Lu—P1B7 IgG (˜650 μCi/mouse, dosed on day (and day 7), and a Sotorasib (30 mg/kg)+¹⁷⁷Lu—P1B7 IgG (˜650 μCi per mouse, dosed on day 0 and day 7) arm. UMUC3 cells were injected into the left flank and tumors grown to ˜200 mm³ volume before study initiation. Mice received either saline or Sotorasib (30 mg/kg) via oral gavage starting on Day 0 and once a day thereafter for two weeks. Mice receiving the ¹⁷⁷Lu—P1B7 IgG were given ˜650 μCi via tail vein injection on Day 1 and Day 7 (FIG. 6A). Tumor size was monitored via caliper once every two days and was reported as fold change relative to initiation of the study.

Untreated xenografts readily grew to 7-fold their original size within two weeks, and those treated with either Sotorasib or ¹⁷⁷Lu—P1B7 IgG as single agents showed a nearly identical growth rate, confirming that this dose of Sotorasib and the ¹⁷⁷Lu—P1B7 IgG alone were ineffective as monotherapeutic agents in this UMUC3 xenograft model. However, combination of Sotorasib with ¹⁷⁷Lu—P1B7 IgG resulted in a significant reduction of UMUC3 tumor growth throughout the entire two-week study (FIG. 6B). The fold change in tumor volume from Day 1 to Day 11 was quantified and found to be significantly smaller (P<0.01) in the Sotorasib (30 mg/kg)+¹⁷⁷Lu—P1B7 IgG combination arm compared to all three of the other arms (FIG. 6C). Additionally, mouse body weight was recorded throughout the study, with no significant differences found between the four treatment arms, confirming that ¹⁷⁷Lu—P1B7 IgG as both a monotherapy and a combination therapy was well tolerated at this dose (FIG. 6D).

The interest in developing and clinically implementing alpha-emitting radioisotopes has grown substantially in the past decade with a growing body of mouse and human data suggesting them to be more effective anticancer payloads compared to beta emitting radioisotopes. Moreover, alpha emitting radioisotopes may be more appropriate for low abundance tumor antigens, given their higher linear energy transfer. On this basis, we next tested if Ac-225 labeled P1B7 IgG can suppress UMUC3 tumor growth when co-administered with Sotorasib. P1B7 IgG was coupled to the chelator Macropa via stochastic modification of lysine side chains and coupled to Ac-225 in >90% radiochemical yield and >99% radiochemical purity (FIG. 9C). ²²⁵Ac—P1B7 was administered either as a monotherapy or in combination with Sotorasib (30 mg/kg) to mice at a dose of 0.6 μCi/mouse, a dose approximately 1,000-fold lower than that used in the Lutetium study. Sotorasib dosing was carried out as previously described, but only a single dose of ²²⁵Ac—P1B7 was administered on Day 1.

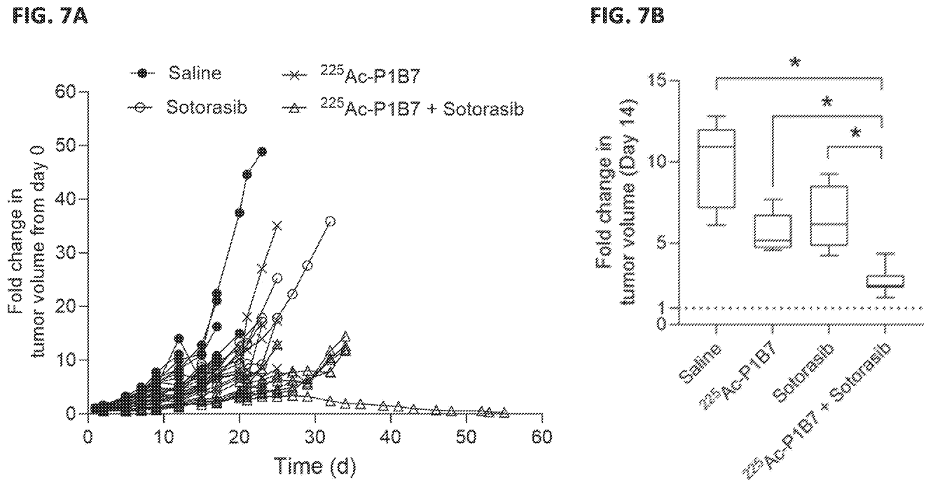

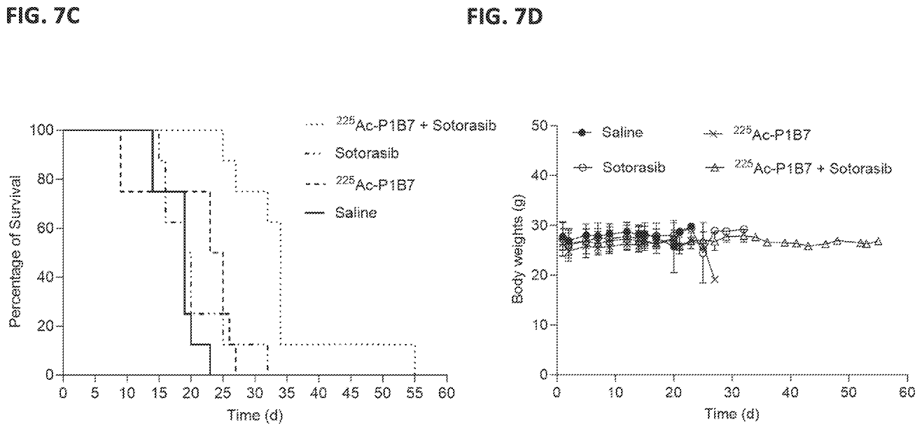

The ²²⁵Ac—P1B7 plus Sotorasib treatment significantly suppressed tumor growth compared to vehicle or either monotherapy (P<0.01) as shown in FIG. 7A-7B. Overall, mice treated with Sotorasib and the ²²⁵Ac—P1B7 IgG survived longer than those treated with either monotherapy (FIG. 7C). Additionally, body weight was monitored throughout the study, and no significant differences were found in any of the treatment arms, indicated that the ²²⁵Ac—P1B7 IgG radioligand therapeutic was well tolerated (FIG. 7D). Collectively, these data provide compelling evidence of in vivo efficacy of an IgG radioligand therapeutic targeting a covalent inhibitor-modified, oncoprotein-derived MHC I complex.

### Example 4. Development of Antibodies Targeting Sotorasib-Labeled MHC I Complexes for Immunohistochemistry

In addition to therapeutic development, Sotorasib-specific antibodies are contemplated to have value for the development of prognostic tests. To determine whether a Sotorasib-specific antibody could be used to predict response to a radioligand therapy like those described above, an immunohistochemistry (IHC) approach using the P1E5 antibody was developed. P1E5 was labeled with Digoxigenin (Biocare) and an anti-Dig detection cascade was used on the Ventana Discovery Ultra platform. H2122 cells (KRas G12C) were either treated with 1 μM Sotorasib or left untreated for 48 hours before formalin fixing. Antigen retrieval was conducted in citrate buffer (pH=6) for 16 or 32 minutes. The P1E5 primary IgG was incubated for 1 hour (RT), followed by 16 minutes for anti-Dig IgG binding (RT), and 32 minutes of subsequent linker incubation before detection via horseradish peroxidase activity (37° C.).

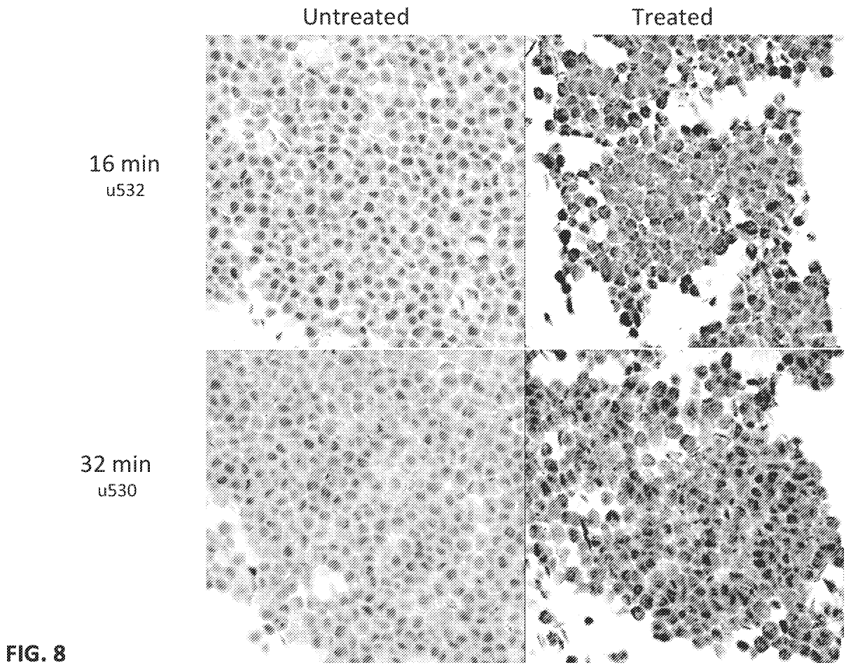

P1E5 readily stained the Sotorasib treated H2122 cells but not the untreated cells, confirming the Sotorasib-dependency of the staining (FIG. 8). This observation demonstrates that the antibodies described herein are amenable to IHC applications, and is indicative of their suitability for use in characterizing patient samples.

ADDITIONAL SEQUENCES
Anti-ARS-1620 Light Chain & Heavy Chain Sequences
SEQ ID NO: 5
>P1A4 LC
LFAIPLVVPFYSHSAQSVVTQPPSVSAAPGQEVTISCSGSSSNIGNNYVSWYQHLPGTAP
KILIYDSDKRPSGIPDRESGSKSGTSATLGVTGLQTGDEADYYCGTWDSSLSAVMFGGGT
KLTVLGQPKAAPSVTLFPPSSEELQANKATLVCLISDFYPGAVTVAWKADSSPVKAGVET
TTPSKQSNNKYAASSYLSLTPEQWKSHKSYSCQVTHEGSTVEKTVAPTECS
SEQ ID NO: 6
>P2B2 LC
LFAIPLVVPFYSHSAQSVVTQPPSVSAAPGHKVSISCSGSNSNIGHNYVSWYQQFPGTAPKLLV
YRDDNRPSEVPDRFSGSKSGTSATLGITGLQTADEADYYCGTWDSTLQHVVEGGGTKLTVLSQP
KAAPSVTLFPPSSEELQANKATLVCLISDFYPGAVTVAWKADSSPVKAGVETTTPSKQSNNKYA
ASSYLSLTPEQWKSHKSYSCQVTHEGSTVEKTVAPTECS
SEQ ID NO: 7
>P1C10 LC
IFAIPLVVPFYSHSAQSVVTQPPSVSAAPGQKVTISCSGSSSNIGNHYVSWYQQLPGTAPKLLI
YDDGKRPSGIPDRFSGSKSGTSATLGITGLQTGDEADYYCATWDNSLSVVLFGGGTKLTVLGQP
KAAPSVTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECS
SEQ ID NO: 8
>P1A4 HC
QVQLQESGGGLVQPGRSLRLSCAASGFTESSYAMHWVRQAPGKGLEWVAVISYDGSNKYY
ADSVKGRATISRDNSKNTLYLQMNSLRAEDTAVYYCARDYDWQYFDYWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCAAA
SEQ ID NO: 9
>P2B2 HC
QVQLVESGGGVVQPGRYXRLSCATSGFTFSNYAMHWVRQAPGKGLEWVALISYDGSNKYYADSV
KGRFTISRDKSTNALYLQMNSLRVEDTAVYYCARERVPWDYYGMDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAA
SEQ ID NO: 10
>P1C10 HC
QVQLVQSGGGLVKPGGSLRLSCAASGFTFSSYSMNWVRQAPGKGLEWVSYISSRGRTIYYADSV
KGRFTISRDNAKNSLYLQMNSLRAEDTAVYYCVRDVPGVWGFAYWGQGTLVTVSSASTKGPSVE
PLAPSSKSTSGGTAALGCLVKDYFPEPVTVSWNSGALISGVHTFPAVLQSSGLYSLSSVVTVPS
SSIGTQTYICNVNHKPSNTKVDKKVEPKSCAAA
Anti-AMG 510 (sotorasib) Light Chain & Heavy Chain Sequences
SEQ ID NO: 11
>P1B7 LC
LFAIPLVVPFYSHSAQSALTQPPSVSGSPGQSVTISCTGSSSDVGHYNRVSWYQQSPGTAPKLMIYDVSN
RPSGAFSRFSGSRSGNTASLTISGLQPEDEADYYCSSHRTGYTVEVFGTGTKVTVLGQPKANPTVTLFPP
SSEELQANKATEVCLISDFYPGAVTVAWKADGSPVKAGVETTKPSKQSNNKYAASSYLSLTPEQWKSHRS
YSCQVTHEGSTVEKTVAPAECS
SEQ ID NO: 12
>P1H4 LC
LFAIPLVVPFYSHSALETTLTQSPGTLSLSPGERATLSCRASQSVSSSYLAWYQQKPGQAPRLLIYGASS
RATGIPDRFSGSGSGTDFTLTISRLEPGDFAVYYCQQYGTSPITFGQGTRLEIKRIVAAPSVFIFPPSDE
QLKSGTASVVCLLNNFYPREAKVQWKVDNALQSGNSQESVTEQDSKDSTYSLSSTLTLSKADYEKHKVYA
CEVTHQGLSSPVTKSFNRGEC
SEQ ID NO: 13
>P2B6 LC
LFAIPLVVPFYSHSALDVVMTQSPLSLPVTPGEPASISCRSSQSLLRNNGYNYLDWYLQKPGQSPQLLIY
LGSNRASGVPDRFSGSGSGRDFTLKISRVEAEDVGIYYCMQGAHWPYTFGQGTNLEISRTVAAPSVFIFP
PSDEQLKSGTASVVCLINNFYPREAKVQWKVDNALQSGNSQESVTEQDSKDSTYSLSSTLTLSKADYEKH
KVYACEVTHQGLSSPVTKSFNRGEC
SEQ ID NO: 14
>P2E3 LC
LFAIPLVVPFYSHSALDVVMTQSPLSLPVTPGEAASISCRSSQSLLHSNGYTYLDWYLQKPGQSPRLLIY
SGSNRASGVPDRFSGSGSGTDFTLKISRVEAEDVGVYYCMQGTHWPYTFGQGTRLEIKRTVAAPSVFIFP
PSDEQLKSGTASVVCLLNNFYPREAKVQWKVDNALQSGNSQESVTEQDSKDSTYSLSSTLTLSKADYEKH
KLYACEVTHQGLSSPVTKSFNRGEC
SEQ ID NO: 15
>P1E5 LC
LFAIPLVVPFYSHSALDVVMTQSPLSLPVTPGEPASISCRSSQSLLHSNGYNYLDWYLQKPGQSPQLLIY
LGSNRASGVPDRFSGSGSGTDFTLKISRVEAEDVGVYYCMQGTHWPPGYTFGQGTKVEINRTVAAPSVFI
FPPSDEQLKSGTASVVCLINNFYPREAKVQWKVDNALQSGNSQESVTEQDSKDSTYSLSSTLTLSKADYE
KHKVYACEVTHQGLSSPVTKSFNRGEC
SEQ ID NO: 16
>P2C1 LC
LFAIPLVVPFYSHSAQSALTQPASVSGSPGQSITISCTGTSSDVGSYNLVSWYQQHPGKAPKLMIYDVSN
RPSGVSNRFSGSKSGNTASLTISGLQAEDEADYYCSSYTSSSTLVFGTGTNVTVLGQPKANPTVTLFPPS
SEELQANKATIVCLISDFYPGAVTVAWKADGSPVKAGVETTKPSKQSNNKYAASSYLSLTPEQWKSHRSY
SCQVTHEGSTVEKTVAPTECS
SEQ ID NO: 17
>P1B7 HC
QVQLQQSGPGLVKPSQTLSLTCAISGDSVSSNSVAWNWIRQSPSRGLEWLGRTYYRSKWYSDYAVSVKSR
ITINPDTSKNQFSLQLTSMTPEDTAVYYCAREGVTMGPGAFDIWGQGTMVTVSSASTKGPSVFPLAPSSK
STSGGTAALGCLVKDYFPEPVTVSWNSGALISGVHTFPAVLQSSGLYSLSSVVTVPSSSLGTQTYICNVN
HKPSNTKVDKKVEPKSCAAA
SEQ ID NO: 18
>P1H4 HC
EVQLVQSGAEVKKPGSSVKVSCKASGGTFSSYAISWVRQAPGQGLEWMGGIIPIFGTANYAQKFQGRVTI
TADKSTSTAYMELSSLRSEDTAVYYCAFNYYDSSGYSDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSCAAA
SEQ ID NO: 19
>P2B6 HC
QLQLQESGPGLVKPSETLSLTCTVSGVSISSRSDHWGWVRQPPGKGLEWIGSISYSGSTYYNPSLKSRVT
ISVDTSKNQLSLKLTSVTAADTAVYYCARRALAQQLVMDVWGQGTTVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCAAA
SEQ ID NO: 20
>P2E3 HC
QVQLQESGPGLVKPSETLSLTCTVSGGSVSSSSYYWGWIRQPPGEGLEWIGSIYYSGSTYYNPSLKSRVT
ISVDTSKNQFSLKLSSVTAADTAVYYCATRERGSFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCAAA
SEQ ID NO: 21
>P1E5 HC
QVQLQESGGGVVQPGRSIRLSCAASGFTFSSYEMNWVRQAPGKGLEWVAVISYDGSNKYYADSVKGRETI
SRDNSKNTLYLQMNSLRAEDTAVYYCARDAYSGWFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCAAA
SEQ ID NO: 22
>P2C1 HC
EVQLVQSGAEVKKPGSSVKVSCKASGGTFSSYAISWVRQAPGQGLEWMGIINPSGGSTSYAQKFQGRVTM
TRDTSTSTVYMELSSLRSEDTAVYYCARDLGTIFEANGAFDIWGQGTMVTVSSASTKGPSVFPLAPSSKS
TSGGTAALGCLVKDYFPEPVTVSWNSGALTSGVHTFPAVLQSSGLYSLSSVVTVPSSSLGTQTYICNVNH
KPSNTKVDKKVEPKSCAAA

1. An antibody or antigen binding fragment thereof, wherein the antibody or fragment binds to an antigen comprising the amino acid sequence VVVGAC (510) GVGK (SEQ ID NO: 100), wherein 510 is AMG-510 (sotorasib) and the antibody or fragment comprises:
(a) a light chain variable region comprising a CDRL1 of SEQ ID NO:49, a CDRL2 of SEQ ID NO:50, and a CDRL3 of SEQ ID NO:51, and a heavy chain variable region comprising a CDRH1 of SEQ ID NO:52, a CDRH2 of SEQ ID NO:53, and a CDRH3 of SEQ ID NO:54;
(b) a light chain variable region comprising a CDRL1 of SEQ ID NO:57, a CDRL2 of SEQ ID NO:58, and a CDRL3 of SEQ ID NO:59, and a heavy chain variable region comprising a CDRH1 of SEQ ID NO:60, a CDRH2 of SEQ ID NO:61, and a CDRH3 of SEQ ID NO:62;
(c) a light chain variable region comprising a CDRL1 of SEQ ID NO:65, a CDRL2 of SEQ ID NO:66, and a CDRL3 of SEQ ID NO:67, and a heavy chain variable region comprising a CDRH1 of SEQ ID NO:68, a CDRH2 of SEQ ID NO:69, and a CDRH3 of SEQ ID NO:70;
(d) a light chain variable region comprising a CDRL1 of SEQ ID NO:73, a CDRL2 of SEQ ID NO:74, and a CDRL3 of SEQ ID NO:75, and a heavy chain variable region comprising a CDRH1 of SEQ ID NO:76, a CDRH2 of SEQ ID NO:77, and a CDRH3 of SEQ ID NO:78;
(e) a light chain variable region comprising a CDRL1 of SEQ ID NO:81, a CDRL2 of SEQ ID NO:82, and a CDRL3 of SEQ ID NO:83, and a heavy chain variable region comprising a CDRH1 of SEQ ID NO:84, a CDRH2 of SEQ ID NO:85, and a CDRH3 of SEQ ID NO:86; or
(f) a light chain variable region comprising a CDRL1 of SEQ ID NO:89, a CDRL2 of SEQ ID NO:90, and a CDRL3 of SEQ ID NO:91, and a heavy chain variable region comprising a CDRH1 of SEQ ID NO:92, a CDRH2 of SEQ ID NO:93, and a CDRH3 of SEQ ID NO:94.

2. The antibody or fragment of claim 1, wherein the antibody or fragment is human or humanized.

3. The antibody or fragment of claim 1, wherein:
(a) the light chain variable region comprises the amino acid sequence of SEQ ID NO:47 and the heavy chain variable region comprises the amino acid sequence of SEQ ID NO:48;
(b) the light chain variable region comprises the amino acid sequence of SEQ ID NO:55 and the heavy chain variable region comprises the amino acid sequence of SEQ ID NO:56;
(c) the light chain variable region comprises the amino acid sequence of SEQ ID NO:63 and the heavy chain variable region comprises the amino acid sequence of SEQ ID NO:64;
(d) the light chain variable region comprises the amino acid sequence of SEQ ID NO:71 and the heavy chain variable region comprises the amino acid sequence of SEQ ID NO:72;
(e) the light chain variable region comprises the amino acid sequence of SEQ ID NO:79 and the heavy chain variable region comprises the amino acid sequence of SEQ ID NO:80; or
(f) the light chain variable region comprises the amino acid sequence of SEQ ID NO:87 and the heavy chain variable region comprises the amino acid sequence of SEQ ID NO:88.

4. The antibody or fragment of claim 3, comprising:
(a) a light chain comprising the amino acid sequence of SEQ ID NO:11 and a heavy chain comprising the amino acid sequence of SEQ ID NO:17, or a light chain comprising an amino acid sequence having at least 95% sequence identity to SEQ ID NO:11 and a heavy chain comprising an amino acid sequence having at least 95% sequence identity to SEQ ID NO:17;
(b) a light chain comprising the amino acid sequence of SEQ ID NO: 12 and a heavy chain comprising the amino acid sequence of SEQ ID NO:18, or a light chain comprising an amino acid sequence having at least 95% sequence identity to SEQ ID NO: 12 and a heavy chain comprising an amino acid sequence having at least 95% sequence identity to SEQ ID NO:18;
(c) a light chain comprising the amino acid sequence of SEQ ID NO:13 and a heavy chain comprising the amino acid sequence of SEQ ID NO:19, or a light chain comprising an amino acid sequence having at least 95% sequence identity to SEQ ID NO: 13 and a heavy chain comprising an amino acid sequence having at least 95% sequence identity to SEQ ID NO:19;
(d) a light chain comprising the amino acid sequence of SEQ ID NO: 14 and a heavy chain comprising the amino acid sequence of SEQ ID NO:20, or a light chain comprising an amino acid sequence having at least 95% sequence identity to SEQ ID NO:14 and a heavy chain comprising an amino acid sequence having at least 95% sequence identity to SEQ ID NO:20;
(e) a light chain comprising the amino acid sequence of SEQ ID NO:15 and a heavy chain comprising the amino acid sequence of SEQ ID NO:21, or a light chain comprising an amino acid sequence having at least 95% sequence identity to SEQ ID NO:15 and a heavy chain comprising an amino acid sequence having at least 95% sequence identity to SEQ ID NO:21; or
(f) a light chain comprising the amino acid sequence of SEQ ID NO:16 and a heavy chain comprising the amino acid sequence of SEQ ID NO:22, or a light chain comprising an amino acid sequence having at least 95% sequence identity to SEQ ID NO:16 and a heavy chain comprising an amino acid sequence having at least 95% sequence identity to SEQ ID NO:22.

5. The antibody or fragment of claim 1, comprising:
(i) a light chain variable region comprising a CDRL1 of SEQ ID NO:49, a CDRL2 of SEQ ID NO:50, and a CDRL3 of SEQ ID NO:51, and a heavy chain variable region comprising a CDRH1 of SEQ ID NO:52, a CDRH2 of SEQ ID NO:53, and a CDRH3 of SEQ ID NO:54;
(ii) a light chain variable region comprising the amino acid sequence of SEQ ID NO:47 and a heavy chain variable region comprising the amino acid sequence of SEQ ID NO:48; or
(iii) a light chain comprising the amino acid sequence of SEQ ID NO:11 and a heavy chain comprising the amino acid sequence of SEQ ID NO:17, or a light chain comprising an amino acid sequence having at least 95% sequence identity to SEQ ID NO:11 and a heavy chain comprising an amino acid sequence having at least 95% sequence identity to SEQ ID NO:17.

6. The antibody or fragment of claim 1, wherein the antibody or fragment is a fragment selected from the group consisting of a Fab, F(ab′)2, Fv and Sfv.

7. The antibody or fragment of claim 1, wherein the antibody or fragment is a full-length human immunoglobulin g (IgG) antibody.

8-10. (canceled)

11. An immunoconjugate comprising the antibody or fragment of claim 1 and a detectable marker or cytotoxic agent.

12. The immunoconjugate of claim 11, wherein the immunoconjugate comprises a detectable marker selected from the group consisting of a radioisotope, a metal chelator, an enzyme, a fluorescent compound, a bioluminescent compound, and a chemiluminescent compound.

13. The immunoconjugate of claim 11, wherein the immunoconjugate comprises a cytotoxic agent selected from the group consisting of an alkylating agent, an antimetabolite, an mitotic inhibitor, an antineoplastic antibiotic, a radionuclide, and a toxin.

14. An isolated nucleic acid encoding the antibody or fragment of claim 1.

15. An expression cassette comprising the nucleic acid of claim 14, in operable combination with a regulatory sequence.

16. A host cell comprising the expression cassette of claim 15 or an expression vector comprising the expression cassette.

17. A method for the production of a recombinant antibody or fragment thereof, the method comprising:
a) culturing the host cell of claim 16 under conditions suitable for expression of the antibody or fragment; and
b) recovering the antibody or fragment from the host cell or its cell culture supernatant.

18. A method for characterizing a biopsy sample from a patient, comprising:
a) contacting the sample with the antibody or fragment of claim 1; and
b) detecting the binding of the antibody or fragment to cells of the sample, wherein the patient has received an effective amount of sotorasib (AMG-510).

19. A method for treating cancer, the method comprising:
administering to a patient with cancer an effective amount of the antibody or fragment of claim 1, wherein the patient has received or is receiving an effective amount of sotorasib (AMG-510).

20. (canceled)

21. The method of claim 19, wherein the cancer is a carcinoma.

22. The method of claim 21, wherein the carcinoma is a non-small cell lung carcinoma (NSCLC), a colorectal adenocarcinoma (CRC), or a pancreatic adenocarcinoma.

23. The method of claim 19, wherein a kirsten rat sarcoma virus homolog (Kras) with a G12C mutation is expressed in cells of the cancer.

24. (canceled)

25. An antibody or antigen binding fragment thereof, wherein the antibody or fragment binds to an antigen comprising the amino acid sequence GAC (1620) GVGKSAL (SEQ ID NO: 2), wherein 1620 is ARS-1620 and the antibody or fragment comprises:
(a) a light chain variable region comprising a CDRL1 of SEQ ID NO:33, a CDRL2 of SEQ ID NO:34, and a CDRL3 of SEQ ID NO:35, and a heavy chain variable region comprising a CDRH1 of SEQ ID NO:36, a CDRH2 of SEQ ID NO:37, and a CDRH3 of SEQ ID NO:38; or
(b) a light chain variable region comprising a CDRL1 of SEQ ID NO:41, a CDRL2 of SEQ ID NO:42, and a CDRL3 of SEQ ID NO:43, and a heavy chain variable region comprising a CDRH1 of SEQ ID NO:44, a CDRH2 of SEQ ID NO:45, and a CDRH3 of SEQ ID NO:46.

26-70. (canceled)


SEQUENCE LISTING (101 sequences)

SEQ ID NO: 1 - protein, 10 aa, synthetic construct
GACGVGKSAL

SEQ ID NO: 2 - protein, 10 aa, synthetic construct
  MOD_RES 3: note = ARS-1620(S-atropisomer)-modified cysteine
GACGVGKSAL

SEQ ID NO: 3 - protein, 10 aa, synthetic construct
  MOD_RES 3: note = ARS-1620(R-atropisomer)-modified cysteine
GACGVGKSAL

SEQ ID NO: 4 - protein, 10 aa, synthetic construct
  MOD_RES 3: note = ARS-1620(S-atropisomer)-modified cysteine
  MOD_RES 10: note = Norleucine
GGCGGGGGGL

SEQ ID NO: 5 - protein, 231 aa, synthetic construct
LFAIPLVVPFYSHSAQSVVTQPPSVSAAPGQEVTISCSGSSSNIGNNYVSWYQHLPGTAPKLLIYDSDKRPSGIPDRFSGSKSGTSATLGVTGLQTGDEADYYCGTWDSSLSAVMFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 6 - protein, 231 aa, synthetic construct
LFAIPLVVPFYSHSAQSVVTQPPSVSAAPGHKVSISCSGSNSNIGHNYVSWYQQFPGTAPKLLVYRDDNRPSEVPDRFSGSKSGTSATLGITGLQTADEADYYCGTWDSTLQHVVFGGGTKLTVLSQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 7 - protein, 231 aa, synthetic construct
LFAIPLVVPFYSHSAQSVVTQPPSVSAAPGQKVTISCSGSSSNIGNHYVSWYQQLPGTAPKLLIYDDGKRPSGIPDRFSGSKSGTSATLGITGLQTGDEADYYCATWDNSLSVVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 224 aa, synthetic construct
QVQLQESGGGLVQPGRSLRLSCAASGFTFSSYAMHWVRQAPGKGLEWVAVISYDGSNKYYADSVKGRFTISRDNSKNTLYLQMNSLRAEDTAVYYCARDYDWQYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 9 - protein, 227 aa, synthetic construct
QVQLVESGGGVVQPGRYXRLSCATSGFTFSNYAMHWVRQAPGKGLEWVALISYDGSNKYYADSVKGRFTISRDKSTNALYLQMNSLRVEDTAVYYCARERVPWDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 10 - protein, 225 aa, synthetic construct
QVQLVQSGGGLVKPGGSLRLSCAASGFTFSSYSMNWVRQAPGKGLEWVSYISSRGRTIYYADSVKGRFTISRDNAKNSLYLQMNSLRAEDTAVYYCVRDVPGVWGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 11 - protein, 232 aa, synthetic construct
LFAIPLVVPFYSHSAQSALTQPPSVSGSPGQSVTISCTGSSSDVGHYNRVSWYQQSPGTAPKLMIYDVSNRPSGAFSRFSGSRSGNTASLTISGLQPEDEADYYCSSHRTGYTVEVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPAECS

SEQ ID NO: 12 - protein, 231 aa, synthetic construct
LFAIPLVVPFYSHSALETTLTQSPGTLSLSPGERATLSCRASQSVSSSYLAWYQQKPGQAPRLLIYGASSRATGIPDRFSGSGSGTDFTLTISRLEPGDFAVYYCQQYGTSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 235 aa, synthetic construct
LFAIPLVVPFYSHSALDVVMTQSPLSLPVTPGEPASISCRSSQSLLRNNGYNYLDWYLQKPGQSPQLLIYLGSNRASGVPDRFSGSGSGRDFTLKISRVEAEDVGIYYCMQGAHWPYTFGQGTNLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 235 aa, synthetic construct
LFAIPLVVPFYSHSALDVVMTQSPLSLPVTPGEAASISCRSSQSLLHSNGYTYLDWYLQKPGQSPRLLIYSGSNRASGVPDRFSGSGSGTDFTLKISRVEAEDVGVYYCMQGTHWPYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 15 - protein, 237 aa, synthetic construct
LFAIPLVVPFYSHSALDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPPGYTFGQGTKVEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 231 aa, synthetic construct
LFAIPLVVPFYSHSAQSALTQPASVSGSPGQSITISCTGTSSDVGSYNLVSWYQQHPGKAPKLMIYDVSNRPSGVSNRFSGSKSGNTASLTISGLQAEDEADYYCSSYTSSSTLVFGTGTNVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 230 aa, synthetic construct
QVQLQQSGPGLVKPSQTLSLTCAISGDSVSSNSVAWNWIRQSPSRGLEWLGRTYYRSKWYSDYAVSVKSRITINPDTSKNQFSLQLTSMTPEDTAVYYCAREGVTMGPGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 18 - protein, 226 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAFNYYDSSGYSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 19 - protein, 227 aa, synthetic construct
QLQLQESGPGLVKPSETLSLTCTVSGVSISSRSDHWGWVRQPPGKGLEWIGSISYSGSTYYNPSLKSRVTISVDTSKNQLSLKLTSVTAADTAVYYCARRALAQQLVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 20 - protein, 224 aa, synthetic construct
QVQLQESGPGLVKPSETLSLTCTVSGGSVSSSSYYWGWIRQPPGEGLEWIGSIYYSGSTYYNPSLKSRVTISVDTSKNQFSLKLSSVTAADTAVYYCATRLRGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 21 - protein, 224 aa, synthetic construct
QVQLQESGGGVVQPGRSLRLSCAASGFTFSSYEMNWVRQAPGKGLEWVAVISYDGSNKYYADSVKGRFTISRDNSKNTLYLQMNSLRAEDTAVYYCARDAYSGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 22 - protein, 229 aa, synthetic construct
EVQLVQSGAEVKKPGSSVKVSCKASGGTFSSYAISWVRQAPGQGLEWMGIINPSGGSTSYAQKFQGRVTMTRDTSTSTVYMELSSLRSEDTAVYYCARDLGTIFEANGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 23 - protein, 110 aa, synthetic construct
QSVVTQPPSVSAAPGQEVTISCSGSSSNIGNNYVSWYQHLPGTAPKLLIYDSDKRPSGIPDRFSGSKSGTSATLGVTGLQTGDEADYYCGTWDSSLSAVMFGGGTKLTVL

SEQ ID NO: 24 - protein, 118 aa, synthetic construct
QVQLQESGGGLVQPGRSLRLSCAASGFTFSSYAMHWVRQAPGKGLEWVAVISYDGSNKYYADSVKGRFTISRDNSKNTLYLQMNSLRAEDTAVYYCARDYDWQYFDYWGQGTLVTVSS

SEQ ID NO: 25 - protein, 13 aa, synthetic construct
SGSSSNIGNNYVS

SEQ ID NO: 26 - protein, 7 aa, synthetic construct
DSDKRPS

SEQ ID NO: 27 - protein, 11 aa, synthetic construct
GTWDSSLSAVM

SEQ ID NO: 28 - protein, 10 aa, synthetic construct
GFTFSSYAMH

SEQ ID NO: 29 - protein, 10 aa, synthetic construct
VISYDGSNKY

SEQ ID NO: 30 - protein, 8 aa, synthetic construct
DYDWQYFD

SEQ ID NO: 31 - protein, 110 aa, synthetic construct
QSVVTQPPSVSAAPGHKVSISCSGSNSNIGHNYVSWYQQFPGTAPKLLVYRDDNRPSEVPDRFSGSKSGTSATLGITGLQTADEADYYCGTWDSTLQHVVFGGGTKLTVL

SEQ ID NO: 32 - protein, 121 aa, synthetic construct
QVQLVESGGGVVQPGRYLRLSCATSGFTFSNYAMHWVRQAPGKGLEWVALISYDGSNKYYADSVKGRFTISRDKSTNALYLQMNSLRVEDTAVYYCARERVPWDYYGMDVWGQGTTVTVSS

SEQ ID NO: 33 - protein, 13 aa, synthetic construct
SGSNSNIGHNYVS

SEQ ID NO: 34 - protein, 7 aa, synthetic construct
RDDNRPS

SEQ ID NO: 35 - protein, 11 aa, synthetic construct
GTWDSTLQHVV

SEQ ID NO: 36 - protein, 10 aa, synthetic construct
GFTFSNYAMH

SEQ ID NO: 37 - protein, 11 aa, synthetic construct
LISYDGSNKYY

SEQ ID NO: 38 - protein, 12 aa, synthetic construct
ERVPWDYYGMDV

SEQ ID NO: 39 - protein, 110 aa, synthetic construct
QSVVTQPPSVSAAPGQKVTISCSGSSSNIGNHYVSWYQQLPGTAPKLLIYDDGKRPSGIPDRFSGSKSGTSATLGITGLQTGDEADYYCATWDNSLSVVLFGGGTKLTVL

SEQ ID NO: 40 - protein, 119 aa, synthetic construct
QVQLVQSGGGLVKPGGSLRLSCAASGFTFSSYSMNWVRQAPGKGLEWVSYISSRGRTIYYADSVKGRFTISRDNAKNSLYLQMNSLRAEDTAVYYCVRDVPGVWGFAYWGQGTLVTVSS

SEQ ID NO: 41 - protein, 13 aa, synthetic construct
SGSSSNIGNHYVS

SEQ ID NO: 42 - protein, 7 aa, synthetic construct
DDGKRPS

SEQ ID NO: 43 - protein, 11 aa, synthetic construct
ATWDNSLSVVL

SEQ ID NO: 44 - protein, 10 aa, synthetic construct
GFTFSSYSMN

SEQ ID NO: 45 - protein, 11 aa, synthetic construct
YISSRGRTIYY

SEQ ID NO: 46 - protein, 10 aa, synthetic construct
DVPGVWGFAY

SEQ ID NO: 47 - protein, 232 aa, synthetic construct; identical to SEQ ID NO: 11

SEQ ID NO: 48 - protein, 230 aa, synthetic construct; identical to SEQ ID NO: 17

SEQ ID NO: 49 - protein, 14 aa, synthetic construct
TGSSSDVGHYNRVS

SEQ ID NO: 50 - protein, 7 aa, synthetic construct
DVSNRPS

SEQ ID NO: 51 - protein, 11 aa, synthetic construct
SSHRTGYTVEV

SEQ ID NO: 52 - protein, 12 aa, synthetic construct
GDSVSSNSVAWN

SEQ ID NO: 53 - protein, 11 aa, synthetic construct
RTYYRSKWYSD

SEQ ID NO: 54 - protein, 11 aa, synthetic construct
EGVTMGPGAFD

SEQ ID NO: 55 - protein, 231 aa, synthetic construct; identical to SEQ ID NO: 12

SEQ ID NO: 56 - protein, 226 aa, synthetic construct; identical to SEQ ID NO: 18

SEQ ID NO: 57 - protein, 12 aa, synthetic construct
RASQSVSSSYLA

SEQ ID NO: 58 - protein, 7 aa, synthetic construct
GASSRAT

SEQ ID NO: 59 - protein, 9 aa, synthetic construct
QQYGTSPIT

SEQ ID NO: 60 - protein, 10 aa, synthetic construct
GGTFSSYAIS

SEQ ID NO: 61 - protein, 10 aa, synthetic construct
GIIPIFGTAN

SEQ ID NO: 62 - protein, 10 aa, synthetic construct
NYYDSSGYSD

SEQ ID NO: 63 - protein, 235 aa, synthetic construct; identical to SEQ ID NO: 13

SEQ ID NO: 64 - protein, 227 aa, synthetic construct; identical to SEQ ID NO: 19

SEQ ID NO: 65 - protein, 16 aa, synthetic construct
RSSQSLLRNNGYNYLD

SEQ ID NO: 66 - protein, 7 aa, synthetic construct
LGSNRAS

SEQ ID NO: 67 - protein, 9 aa, synthetic construct
MQGAHWPYT

SEQ ID NO: 68 - protein, 12 aa, synthetic construct
GVSISSRSDHWG

SEQ ID NO: 69 - protein, 9 aa, synthetic construct
SISYSGSTY

SEQ ID NO: 70 - protein, 10 aa, synthetic construct
RALAQQLVMD

SEQ ID NO: 71 - protein, 235 aa, synthetic construct; identical to SEQ ID NO: 14

SEQ ID NO: 72 - protein, 224 aa, synthetic construct; identical to SEQ ID NO: 20

SEQ ID NO: 73 - protein, 16 aa, synthetic construct
RSSQSLLHSNGYTYLD

SEQ ID NO: 74 - protein, 7 aa, synthetic construct
SGSNRAS

SEQ ID NO: 75 - protein, 9 aa, synthetic construct
MQGTHWPYT

SEQ ID NO: 76 - protein, 12 aa, synthetic construct
GGSVSSSSYYWG

SEQ ID NO: 77 - protein, 9 aa, synthetic construct
SIYYSGSTY

SEQ ID NO: 78 - protein, 7 aa, synthetic construct
RLRGSFD

SEQ ID NO: 79 - protein, 237 aa, synthetic construct; identical to SEQ ID NO: 15

SEQ ID NO: 80 - protein, 224 aa, synthetic construct; identical to SEQ ID NO: 21

SEQ ID NO: 81 - protein, 16 aa, synthetic construct
RSSQSLLHSNGYNYLD

SEQ ID NO: 82 - protein, 7 aa, synthetic construct
LGSNRAS

SEQ ID NO: 83 - protein, 11 aa, synthetic construct
MQGTHWPPGYT

SEQ ID NO: 84 - protein, 10 aa, synthetic construct
GFTFSSYEMN

SEQ ID NO: 85 - protein, 10 aa, synthetic construct
VISYDGSNKY

SEQ ID NO: 86 - protein, 8 aa, synthetic construct
DAYSGWFD

SEQ ID NO: 87 - protein, 231 aa, synthetic construct; identical to SEQ ID NO: 16

SEQ ID NO: 88 - protein, 229 aa, synthetic construct; identical to SEQ ID NO: 22

SEQ ID NO: 89 - protein, 14 aa, synthetic construct
TGTSSDVGSYNLVS

SEQ ID NO: 90 - protein, 7 aa, synthetic construct
DVSNRPS

SEQ ID NO: 91 - protein, 10 aa, synthetic construct
SSYTSSSTLV

SEQ ID NO: 92 - protein, 10 aa, synthetic construct
GGTFSSYAIS

SEQ ID NO: 93 - protein, 10 aa, synthetic construct
IINPSGGSTS

SEQ ID NO: 94 - protein, 14 aa, synthetic construct
DLGTIFEANGAFDI

SEQ ID NO: 95 - protein, 9 aa, Yellow fever virus
ALNTITNLK

SEQ ID NO: 96 - protein, 10 aa, synthetic construct
KLVVVGACGV

SEQ ID NO: 97 - protein, 10 aa, synthetic construct
  MOD_RES 8: note = ARS-1620(S-atropisomer)-modified cysteine
KLVVVGACGV

SEQ ID NO: 98 - protein, 10 aa, synthetic construct
VVVGACGVGK

SEQ ID NO: 99 - protein, 10 aa, synthetic construct
  MOD_RES 6: note = ARS-1620(S-atropisomer)-modified cysteine
VVVGACGVGK

SEQ ID NO: 100 - protein, 10 aa, synthetic construct
  MOD_RES 6: note = AM510-modified cysteine
VVVGACGVGK

SEQ ID NO: 101 - protein, 9 aa, Influenza virus
GILGFVFTL